SEQUENCE LISTING (79 sequences)

SEQ ID NO: 1 - DNA, 52 bp, artificial sequence
  Synthetic Bi-allelic marker No. 1
ccttcagtcacactaggcttctttcartttattgaatacacaaagcattttg

SEQ ID NO: 2 - DNA, 52 bp, artificial sequence
  Synthetic Bi-allelic marker No. 2
cacatttctcagcaaagctttccaacsaaaatttttatgataatcctttgtc

SEQ ID NO: 3 - DNA, 52 bp, artificial sequence
  Synthetic Bi-allelic marker No. 3
tcatatagaatatgagaatgggaggtmcatctgctgtttcctcacttggtgg

SEQ ID NO: 4 - DNA, 52 bp, artificial sequence
  Synthetic Bi-allelic marker No. 4
ttagtacttacaggctaaatacttaaktaacaagcataaaccagggttatcc

SEQ ID NO: 5 - DNA, 52 bp, artificial sequence
  Synthetic Bi-allelic marker No. 5
cttctattattaatggttctactttgrtaacccttttattattgtgaagctt

SEQ ID NO: 6 - DNA, 52 bp, artificial sequence
  Synthetic Bi-allelic marker No. 6
ttgagaatcaccatgttcaagcatagrcttggctactgagtgagaaatactg

SEQ ID NO: 7 - DNA, 52 bp, artificial sequence
  Synthetic Bi-allelic marker No. 7
acggtacagttcagtggccggggaagytgttttggaagcagaaagaagtgag

SEQ ID NO: 8 - DNA, 52 bp, artificial sequence
  Synthetic Bi-allelic marker No. 8
caccaaaaagtaaacagttttctctaratagtgagaatacaggcaaattttt

SEQ ID NO: 9 - DNA, 52 bp, artificial sequence
  Synthetic Bi-allelic marker No. 9
ggcggccctttggggatcctgtcaggrgtttatactgacccgccatcaatgc

SEQ ID NO: 10 - DNA, 52 bp, artificial sequence
  Synthetic Bi-allelic marker No. 10
gcatagccttttgggtgaggaagttgractaagatttcagagctctcttgct

SEQ ID NO: 11 - DNA, 52 bp, artificial sequence
  Synthetic Bi-allelic marker No. 11
catgtaattatatcttattattaaaastctaccaactcaaagcttccccctt

SEQ ID NO: 12 - DNA, 52 bp, artificial sequence
  Synthetic Bi-allelic marker No. 12
acaagcccgcattcgcccgggtcaggrgctgctctgtgtgaggagcgctgtc

SEQ ID NO: 13 - DNA, 52 bp, artificial sequence
  Synthetic Bi-allelic marker No. 13
agatgctgtctccagaaagcacatgtwaaaggacctatatttgatcagatta

SEQ ID NO: 14 - DNA, 52 bp, artificial sequence
  Synthetic Bi-allelic marker No. 14
gcctttttcttggctaatcttatcatrtttaactaaatgccattttactgtc

SEQ ID NO: 15 - DNA, 52 bp, artificial sequence
  Synthetic Bi-allelic marker No. 15
acagatctgtatttgtatgttgtgtgyatacagtcacagacacacacattca

SEQ ID NO: 16 - DNA, 52 bp, artificial sequence
  Synthetic Bi-allelic marker No. 16
gaccctatcttgattttgaccttgtcrgtaaagcacgggctagcaaaatgcc

SEQ ID NO: 17 - DNA, 52 bp, artificial sequence
  Synthetic Bi-allelic marker No 17
aacaaccagaaaggtgctaggttgcayattacacatttgcactttgtaattt

SEQ ID NO: 18 - DNA, 52 bp, artificial sequence
  Synthetic Bi-allelic marker No 18
gccctgtaatgagatcgctagttctcrtatgcttcaaggtctgaaacagtga

SEQ ID NO: 19 - DNA, 52 bp, artificial sequence
  Synthetic Bi-allelic marker No. 19
agttagtacagacatagtccacattcrgcttctcttaacgttaacatttatt

SEQ ID NO: 20 - DNA, 52 bp, artificial sequence
  Synthetic Bi-allelic marker No. 20
aaccaagtctgattagaacttacaagyaatgaggaaagatgaagctgtagcg

SEQ ID NO: 21 - DNA, 52 bp, artificial sequence
  Synthetic Bi-allelic marker No. 21
ctgcctatgccatggtaaatagttcayttatgaaatctcagggcaactcttt

SEQ ID NO: 22 - DNA, 52 bp, artificial sequence
  Synthetic Bi-allelic marker No. 22
cggatttaaggtaggtgctcaaaaatkgcagttatttttttaaagcctgatt

SEQ ID NO: 23 - DNA, 52 bp, artificial sequence
  Synthetic Bi-allelic marker No. 23
aactggataggaagattaagtcctggsgttgtgtagccctgtaggatgactg

SEQ ID NO: 24 - DNA, 52 bp, artificial sequence
  Synthetic Bi-allelic marker No. 24
gttatccaggttttgttcctttttatytgctctctttttggtgatggtttct

SEQ ID NO: 25 - DNA, 47 bp, artificial sequence
  Synthetic primer
ggggacaactttgtatacaaaagttgatgtcacgtgatagtggcaac

SEQ ID NO: 26 - DNA, 41 bp, artificial sequence
  Synthetic primer
gtgaaagtaggatgagacagctcagatacgcatttcttcac

SEQ ID NO: 27 - DNA, 21 bp, artificial sequence
  Synthetic primer
gctgtctcatcctactttcac

SEQ ID NO: 28 - DNA, 55 bp, artificial sequence
  Synthetic primer
ggggaccactttgtacaagaaagctgggtattatagttcatccatgccatgtgta

SEQ ID NO: 29 - DNA, 52 bp, artificial sequence
  Synthetic primer
ggggacaagtttgtacaaaaaagcaggctcctgcaggtacccggagtagttg

SEQ ID NO: 30 - DNA, 58 bp, artificial sequence
  Synthetic primer
ggggacaacttttgtatacaaagttgtggcgcgccaatgcgcgaaattgtggctactc

SEQ ID NO: 31 - DNA, 21 bp, artificial sequence
  Synthetic primer
ttttgcgtttgctcgtcgcac

SEQ ID NO: 32 - DNA, 24 bp, artificial sequence
  Synthetic primer
agtcgacctgcaggcatgcaagct

SEQ ID NO: 33 - DNA, 24 bp, artificial sequence
  Synthetic primer
agcttgcatgcctgcaggtcgact

SEQ ID NO: 34 - DNA, 27 bp, artificial sequence
  Synthetic primer
ggaaacagttatgtttggtatattggg

SEQ ID NO: 35 - DNA, 20 bp, artificial sequence
  Synthetic primer
tctgtgtcagcagcttttcg

SEQ ID NO: 36 - DNA, 20 bp, artificial sequence
  Synthetic primer
acggccgtccgtaatagata

SEQ ID NO: 37 - DNA, 22 bp, artificial sequence
  Synthetic primer
tgagcaagttgtaatggggtta

SEQ ID NO: 38 - DNA, 21 bp, artificial sequence
  Synthetic primer
catctggcacttatggggtta

SEQ ID NO: 39 - DNA, 20 bp, artificial sequence
  Synthetic primer
aaccaggagcaccattcaac

SEQ ID NO: 40 - DNA, 20 bp, artificial sequence
  Synthetic primer
gcaacttcttcctgctgacc

SEQ ID NO: 41 - DNA, 21 bp, artificial sequence
  Synthetic primer
ggcttttcccagtccttcatt

SEQ ID NO: 42 - DNA, 21 bp, artificial sequence
  Synthetic primer
ttctgaacgcgcatgaagctt

SEQ ID NO: 43 - DNA, 21 bp, artificial sequence
  Synthetic primer
gattgttattaatatcctttt

SEQ ID NO: 44 - DNA, 21 bp, artificial sequence
  Synthetic primer
ttgggtgaagttctatttgtt

SEQ ID NO: 45 - DNA, 21 bp, artificial sequence
  Synthetic primer
ttaccgatcaacatgctagtt

SEQ ID NO: 46 - DNA, 21 bp, artificial sequence
  Synthetic primer
taaaggtgtctcgttcttctt

SEQ ID NO: 47 - DNA, 21 bp, artificial sequence
  Synthetic primer
gcctatagtatgtgcttaatt

SEQ ID NO: 48 - DNA, 20 bp, artificial sequence
  Synthetic primer
tcctgctacctcccagaaga

SEQ ID NO: 49 - DNA, 20 bp, artificial sequence
  Synthetic primer
tgagacctcaaagcagcctc

SEQ ID NO: 50 - DNA, 20 bp, artificial sequence
  Synthetic primer
gtgacgcccgccatttacag

SEQ ID NO: 51 - DNA, 23 bp, artificial sequence
  Synthetic primer
catccctccaatgaccgatttcc

SEQ ID NO: 52 - DNA, 18 bp, artificial sequence
  Synthetic primer
cccttcgcctccctcatc

SEQ ID NO: 53 - DNA, 20 bp, artificial sequence
  Synthetic primer
gtccctgtaagccttcttgg

SEQ ID NO: 54 - DNA, 18 bp, artificial sequence
  Synthetic primer
tggctgacttcgctttgg

SEQ ID NO: 55 - DNA, 21 bp, artificial sequence
  Synthetic primer
tccacaccatgaactacatcc

SEQ ID NO: 56 - DNA, 21 bp, artificial sequence
  Synthetic primer
ctgctgctctcaaggttgttc

SEQ ID NO: 57 - DNA, 22 bp, artificial sequence
  Synthetic primer
ctgcttcttcttccgatagtgc

SEQ ID NO: 58 - DNA, 14 bp, artificial sequence
  Synthetic primer
tttgccgcgagccg

SEQ ID NO: 59 - DNA, 24 bp, artificial sequence
  Synthetic primer
taacctggttcatcatcgctaatc

SEQ ID NO: 60 - DNA, 18 bp, artificial sequence
  Synthetic primer
aactgacacctatgcttg

SEQ ID NO: 61 - DNA, 18 bp, artificial sequence
  Synthetic primer
gttgaagtggatactgca

SEQ ID NO: 62 - DNA, 24 bp, artificial sequence
  Synthetic primer
agcatcttatctcgggtgttacct

SEQ ID NO: 63 - DNA, 15 bp, artificial sequence
  Synthetic primer
ccagccccggtgtcc

SEQ ID NO: 64 - DNA, 18 bp, artificial sequence
  Synthetic primer
ctgttagaacgcgacacc

SEQ ID NO: 65 - DNA, 18 bp, artificial sequence
  Synthetic primer
ctgttagaacacgacacc

SEQ ID NO: 66 - DNA, 20 bp, artificial sequence
  Synthetic primer
acacgccacagatacatagc

SEQ ID NO: 67 - DNA, 19 bp, artificial sequence
  Synthetic primer
agtcaacctcctccaatcc

SEQ ID NO: 68 - DNA, 20 bp, artificial sequence
  Synthetic primer
cacgctgcttctgccaggac

SEQ ID NO: 69 - DNA, 20 bp, artificial sequence
  Synthetic primer
aggcgtcaggagatggaccg

SEQ ID NO: 70 - DNA, 52 bp, artificial sequence
  Synthetic Biallelic marker No. 25
ttcttctaacaggtgaatgccttctcrgtacagagttccctcctctcacagc

SEQ ID NO: 71 - DNA, 52 bp, artificial sequence
  Synthetic Biallelic marker No. 26
ccaatccttacctatggtctcaaaccrctatagacacaaatttagttacaca

SEQ ID NO: 72 - DNA, 52 bp, artificial sequence
  Synthetic Biallelic marker No.27
tttacaattcataagcaatcatttatrtaaaaatctacctggtaaggcatat

SEQ ID NO: 73 - DNA, 52 bp, artificial sequence; identical to SEQ ID NO: 70
  Synthetic Biallelic marker No. 28

SEQ ID NO: 74 - DNA, 52 bp, artificial sequence
  Synthetic Biallelic marker No. 29
ttctaataatccaagggtgcatgcatwaaaaaatgattatttgtgcctaaca

SEQ ID NO: 75 - DNA, 52 bp, artificial sequence
  Synthetic Biallelic marker No. 30
atccagttgcacgagttcgcgccgctsgtggagtacggctgccacggccacc

SEQ ID NO: 76 - DNA, 52 bp, artificial sequence
  Synthetic Biallelic marker No. 31
aaaaagccacaaaatgtagcactgaarggtttctgagggctgggcctcttag

SEQ ID NO: 77 - DNA, 52 bp, artificial sequence
  Synthetic Biallelic marker No. 32
ctgctgcacctttattccaagcaaaasgggaacaaggttgcatggcaaagag

SEQ ID NO: 78 - DNA, 52 bp, artificial sequence
  Synthetic Biallelic marker No. 33
agggaaagacagagaatatttttcackgctgtagaggctctgtgcctgcctt

SEQ ID NO: 79 - DNA, 52 bp, artificial sequence
  Synthetic Biallelic marker No. 34
tctgtaatgtaagaaatcagttaattwaaaaaaaattgcaaaatctccataa

(19) United States
(12) Patent Application Publication
(10) Pub. No.: US 2014/0377379 A1
(43) Pub. Date: Dec. 25, 2014

(54) METHOD FOR PROGNOSING THE AGE-AT-ONSET OF HUNTINGTON'S DISEASE

(71) Applicant: Christian Neri, Paris (FR)

(72) Inventor: Christian Neri, Paris (FR)

(73) Assignee: Institut National de la Sante et de la Recherche Medicale (INSERM), Paris (FR)

(21) Appl. No.: 13/820,286

(22) PCT Filed: Sep. 2, 2011
(86) PCT No.: PCT/EP2011/065203

§ 371 Date: Aug. 7, 2014

(30) Foreign Application Priority Data
Sep. 2, 2010 (EP) 10305945.7

(51) Int. Cl. C12Q 1/68; A61K 33/00

(52) U.S. Cl. CPC C12Q 1/6883; A61K 33/00; C12Q 2600/118; C12Q 2600/112; C12Q 2600/156
USPC 424/722; 435/6.11

(57) ABSTRACT

The present invention relates to the prediction of the age-at-onset of Huntington's disease. More specifically, it relates to the use of at least one FoxO-centered network related biallelic markers, preferably single nucleotide polymorphism (SNP) markers, in particular GSK-3β and/or TCERG1 and/or FOXO1 and/or FZD10 related biallelic markers, for prognosing the age-at-onset of symptoms of Huntington's disease in an individual at risk of suffering from said disease. The invention also relates to lithium or a salt thereof for use at low doses in the treatment or prevention of Huntington's disease

### FIELD OF THE INVENTION

The present invention relates to the prediction of the age-at-onset of Huntington's disease. More specifically, it relates to the use of at least one FoxO-centered network related biallelic markers, preferably single nucleotide polymorphism (SNP) markers, in particular GSK-3β and/or TCERG1 and/or FOXO1 and/or FZD10 related biallelic markers, for prognosing the age-at-onset of symptoms of Huntington's disease in an individual at risk of suffering from said disease. The invention also relates to lithium or a salt thereof for use at low doses in the treatment or prevention of Huntington's disease.

### BACKGROUND

Huntington's disease (HD), also called chorea disorder, is a progressive neurodegenerative genetic disorder which affects muscle coordination and leads to cognitive decline and dementia (Walker et al. 2007 Lancet 369:218-28).

The disease is caused by an autosomal dominant mutation on either of the two copies of a gene located on the short arm of chromosome 4 at 4p16.3, called huntingtin (htt).

HD is one of several diseases which involve a trinucleotide repeat, leading to the presence of a repeated section in the htt gene. The repeat is that of a sequence of three DNA bases, cytosine-adenine-guanine (CAG), (i.e. . . . CAGCAGCAG . . . ), CAG being the triplet which encodes the amino acid glutamine. Thus, the CAG series results in the production of a chain of glutamines known as a polyglutamine tract (or polyQ tract), and the repeated part of the gene is identified as the polyQ region. This causes striatal and cortical degeneration.

Unaffected people have fewer than 36 repeated glutamines in the polyQ region. However, a sequence of 36 or more glutamines results in the production of altered forms of the HTT protein. A correlation is observed between the number of CAG repeats and the seriousness of the disease which accounts for about 60% of the variation in the age-at-onset of HD symptoms: 36-40 repeats result in a reduced-penetrance form of the disease, with a late onset and slower progression of these symptoms. When a larger number of repeats is present, HD has full penetrance and can occur under the age of 20; it is then referred to as juvenile HD.

Quantifying the number of CAG repeats in the htt gene thus allows a first estimate of the putative age-at-onset of the HD symptoms. However, it is not sufficient to provide a definite evaluation. Indeed, the age-at-onset may vary by 30 years between two individuals having the same number of CAG repeats. Of course, an accurate estimate is fundamental for the patients at risk, as much from a psychological point of view as for taking timely care of the symptoms.

At present, there is thus a need for a specific and reliable method of determining the age-at-onset of the first symptoms of Huntington's disease in individuals at risk. In particular, there is a need to identify other genes than htt, which will allow a more accurate prediction of the age-at-onset in particular for individuals carrying the same mutant CAG repeat allele.

Some other proteins than HTT have been proposed to play a role in HD. For example, the polymorphic transcriptional regulator TCERG1 was shown in American patients to modify HD (Holbert et al. 2001 Proc Natl Acad Sci USA 98:1811-1816). It is neuroprotective in models of HD (Arango et al. 2006 J Neurosci 26:4649-4659) and its nematode ortholog modulates longevity (Ghazi et al. 2009 Plos Genet. 5;e1000639). Others proteins were identified as longevity modulators with neuroprotective activities such as the transcription factor daf-16/FoxO. FoxO proteins are essential to stress resistance (Brunet et al. 2004 Science 303:2011-2015), and DAF-16 is required for neuroprotection by increasing the amount of sirtuin sir-2.1 in a nematode model of HD pathogenesis based on expressing a polyQ-expanded htt-like exon 1 in touch receptor neurons (Parker et al. 2005 Nat Genet. 37:349-350, Parker et al. 2001 Proc Natl Acad Sci USA. 98(23):13318-23).

Any efficacious treatment of HD does not yet exist. The available drugs, such as tetrabenazine, haloperidol, pimozide and tiapride, the benzoadepines and the antidepressants are destined to relieve the symptoms such as, respectively, abnormal involuntary movements, anxiety and depression.

Lithium has been proposed for the treatment of HD and other neurodegenerative diseases but at the current dosage it induces numerous secondary effects. Its therapeutic potential for HD and other neurodegenerative diseases is therefore strongly limited by the consideration of the risk/benefit ratio.

There is thus also a need for an efficacious neuroprotective treatment with little or no secondary effects.

### DESCRIPTION OF THE INVENTION

The present invention arises from the unexpected discovery by the inventors that polymorphisms in the genes of the FoxO-centered network allow the age-at-onset of HD to be best predicted compared to the sole use of CAG repeat polymorphisms in the htt gene. This applies to genes of the FoxO-centered network such as, but not only, the GSK-3β, TCERG1, FoxO1 and FDZ10 genes. Their results lead to a method for prognosing the age at onset of first symptoms of HD, in particular for individual carrying the same mutant CAG repeat allele. The inventors have also discovered that disturbing the activity of several genes of the FoxO-centered network is neuroprotective in nematodes and cellular models of HD pathogenesis, thus uncovering a new rationale for protecting HD neurons from the dysfunction and death produced by mutant htt expression. This rationale is to stimulate the mechanisms that are essential to longevity and cell survival as controlled by FoxO proteins, their upstream regulators such as, but not only, GSK-3β/β-catenin signaling and their transcriptional targets such as, but not only, mitochondrial uncoupling proteins. This paradigm applies to reducing the activity of GSK-3B, increasing the activity of FoxO3a, increasing the activity of β-catenin which is known as a GSK-3β target and FoxO partner.

Lithium is a drug which exhibits among other activities that of directly or indirectly inhibiting GSK-3β and it has been proposed as a potential treatment for HD and other neurodegenerative diseases. The inventors have also surprisingly discovered that, even at low doses, lithium can be used to treat the symptoms of Huntington's disease. The inventors have indeed discovered that micromolar to nanomolar concentrations of lithium strongly reduces expanded-polyQ toxicity in C. elegans neurons with no effect on normal polyQ toxicity, an effect that requires the activity of the longevity-promoting gene daf-16/FoxO. This discovery by the inventors has subsequently led to observe that a low dose formulation of lithium strongly protects HD mice from the effects of mutant huntingtin expression at the levels of behaviour and neuropathology.

### Huntington's Disease

Huntington's disease is a hereditary neurodegenerative disease that generally develops slowly in individuals reaching from thirty to forty years of age, though it can begin at any time between childhood and old age. In the United States alone, about 30,000 people have Huntington's disease, while at least 150,000 others have a 50 percent risk of developing the disease and thousands more of their relatives live with the possibility that they, too, might develop it.

According to the invention, the expression “age-at-onset” refers to the age at which an individual acquires, develops, or first experiences a symptom of Huntington's disease.

Preferably, the “age-at-onset” refers to the age at which a clinician provides a clinical diagnostic. The age-at-onset of the symptoms will preferably be based on the clinical assessment of the examiner as proposed in the Unified Huntington's Disease Rating scale (1996 Movement Disorder 11 (2):136-141).

Huntington's disease symptoms are of three kinds: a movement disorder (motor symptoms), dementia (cognitive symptoms), and psychiatric disturbances (psychiatric symptoms). The movement disorder comprises uncontrolled twitching movements which first tend to involve the fingers and toes and then progress to include the whole body as well as voluntary movements in the form of clumsiness, stiffness or trouble with walking. Dementia refers to a gradual loss of intellectual abilities such as memory, concentration, problem solving and judgment. Psychiatric disturbances do not strike every individual with Huntington's disease, but when they do, they usually take the forms of depression, irritability and apathy. The depression and other psychiatric conditions seem to result from damages to the brain and can be debilitating.

According to the invention, the age-at-onset of symptoms of Huntington's disease is the time when the first(s) symptoms of Huntington's disease appear(s), irrespective of whether these first(s) symptoms are motor symptoms, cognitive symptoms or psychiatric symptoms. In a specific embodiment, the invention relates to the prognostic of the age-at-onset of HD among individuals the first(s) symptoms of which are motor symptoms.

The number of CAG repeats in the htt gene allows to determine a time span for the age-at-onset of HD symptoms. Starting therefrom, it is possible to evaluate the average age when the patient may develop the first symptoms, ages above this average corresponding to a high estimate and ages below this average corresponding to a lower estimate.

According to the invention, the age-at-onset of HD symptoms is said to be <<early>> when it is below the estimated average age, preferentially an early age-at-onset according to the invention is comprised within the 20 years, preferentially within the 12 years preceding the average age as estimated by the number of CAG repeats in the htt gene. Conversely, the age-at-onset of HD symptoms is said to be <<late>> when it is above the estimated average age, preferentially a late age-at-onset according to the invention is comprised within the 20 years, preferentially within the 12 years succeeding average age as estimated by the number of CAG repeats in the htt gene.

According to the invention, an individual at risk of suffering from HD is an individual who has in his family, preferentially his close family such as grandparents, parents, brothers or sisters, someone who has been diagnosed as suffering from HD. Alternatively or additionally an individual at risk of suffering from HD according to the invention is an individual who exhibits a symptom such that it could be considered as an HD symptom. Alternatively or additionally an individual at risk of suffering from HD according to the invention is an individual who carries either one or two mutant CAG repeat allele as determined by genetic diagnosis.

The individual is preferably a human individual, more preferably an European.

### FoxO-Centered Network Related Biallelic

As used herein, the term “allelic marker” refers to a readily identifiable genetic element, such as SNP, micro-satellites or repeats, that exists either as a wild-type allele or as one or more mutated allele(s), and that is associated with a genetic trait of interest. Typically, the first identified allele is designated as the original allele whereas other alleles are designated as alternative alleles. A “biallelic” marker” refers to an allelic marker that has two major alleles. Diploid organisms may be homozygous or heterozygous for a given allelic marker.

According to the invention, the biallelic markers preferably correspond to single-nucleotide polymorphisms (SNPs) that occur in a DNA sequence when a single nucleotide -A, T, C, or G- in the genome differs between members of a species or between paired chromosomes in a single individual. Within a population, SNPs can be assigned an allele frequency. The allele with the lowest allele frequency in a particular population is designed as the “rare allele”. At the opposite, the allele which has a higher frequency will be designed as the “non rare allele”. Throughout the present specification, the SNPs are designated by their accession number in the dbSNP database entry (for example available on the ncbi.nlm.nih.gov/snp and ncbi.nlm.nih.gov/projects/SNP/ world wide web sites).

The invention pertains to FoxO-centered network related biallelic markers, i.e. biallelic markers located within a gene playing a role in the FoxO-centered network.

According to the invention, the FoxO-centered network corresponds to the network in which the evolutionarily conserved Forkhead Box 0 (FoxO) family of transcription factors regulates multiple transcriptional targets involved in multiple cellular processes, including longevity, cell survival, proliferation, stress resistance, apoptosis and metabolism. The FoxO-centered network according to the invention comprises all regulators upstream of FoxO, all FOX(O)co-factors as well as all of the genes the expression of which is regulated by FoxO. Members of the FoxO-centered network are notably described in Van der Vos et al. (2001, Antioxid Redox Signal).

In a preferred embodiment, the FoxO-centered network related biallelic markers according to the invention are biallelic markers, more preferably SNP biallelic markers, carried by genes that encode (i) members of pathways that signal onto FoxO such as for example, but not only, sirtuin (notably but not only SIRT1) signaling pathways, the AMP-kinase signaling pathway, the IGF-1/insulin signaling pathway, the JNK pathway, the canonical Wnt signaling pathway or the GSK3-βcatenin pathway, (ii) FoxO co-factors such as for example, but not only, β-catenin proteins and 14-3-3 proteins and (iii) FoxO transcriptional targets (genes whose expression and/or activity is regulated by FOXO protein activity) whether they are established or putative FOXO target genes as tested in biological model systems.

Preferably, the FoxO-centered network related biallelic markers according to the invention are biallelic markers, preferably SNP biallelic markers carried by genes selected from the group consisting of: GSK-3β, TCERG1 (also named CA150), FoxO genes such as FoxO1A (also named FoxO1) and FoxO3A, FOXA1, frizzeld genes such as FZD10, adducin ADD2, β-catenin, superoxide dismutases such as SOD1, SOD2 and SOD3, ankyrin ANK2, PRKAA1 (also named AMP-kinase or AMPK), TCERG1 (also named CA150, TAF2S or Urn1), FYN, SGK1, DKK1, sirtuins such as SIRT1, SIRT2, and SIRT3, uncoupling mitochondrial proteins such as UCP1, UCP2 and UCP4, GABARAPL1, PRKAB1, frizzeld genes such as FZD10, the wnt co-receptor RYK, the kinase AKT1, PIK3R2, and LIG1.

The GSK-3β gene, also referred to as GSK-3 beta or glycogen synthase kinase-3 beta, encodes a glycogen synthase kinase which is a serine/threonine protein kinase, and which mediates the addition of phosphate molecules to some serine and threonine amino acids in specific cellular proteins. The phosphorylation of these proteins by GSK-3β has usually an inhibitory effect.

The human GSK-3β gene is located on chromosome 3 at the 3q13.3 locus, spanning the base pair interval [119,540,170-119,813,264] on the reverse strand as indicated in Ensembl ENSG00000082701, and it encodes gene products as for example shown in Ensembl CODS ID CCDS2996.

TCERG1, also known as Transcription elongation regulator 1, is a protein which is encoded in humans by the TCERG1 gene. It is a nuclear protein which regulates transcriptional elongation and pre-mRNA splicing. It interacts with the hyperphosphorylated C-terminal domain of RNA polymerase II via multiple FF domains, and with the pre-mRNA splicing factor SF1 via a WW domain. Alternative splicing results in multiple transcript variants encoding different isoforms.

The human TCERG1 gene (also named CA150, TAF2S or Urn1) is located on chromosome 5 at the 5q31 locus, spanning the base pair interval [145,826,874-145,891,524] on the forward strand as indicated in Ensembl ENSG00000113649, and it encodes gene products as is for example shown in Ensembl CODS ID CCDS4282 or Ensembl CODS ID CCDS43379.

The human FoxOA1 (FoxO1) gene is located on chromosome 13 spanning the base pair interval [41,129,817-41,240,734] on the reverse strand as indicated in Ensembl ENSG00000150907, and it encodes gene products as is for example shown in Ensembl CCDS9371.

The human FZD10 gene is located on chromosome 12 spanning the base pair interval [130,647,032-130,650,284] on the forward strand as indicated in Ensembl ENSG00000111432, and it encodes gene products as is for example shown in Ensembl CCDS9267.

The human ADD2 gene is located on chromosome 2 spanning the base pair interval [70,834,750-70,995,357] on the reverse strand as indicated in Ensembl ENSG00000075340, and it encodes gene products as is for example shown in Ensembl CCDS1906, CCDS1909, CCDS46317 or CCDS46318, CCDS46319.

The human CTNNB1 gene, which encodes β-catenin is located on chromosome 3 spanning the base pair interval [41,236,328-41,301,587] on the forward strand as indicated in Ensembl ENSG00000168036, and it encodes gene products as is for example shown in Ensembl CCDS2694.

The human SOD1 gene is located on chromosome 21 spanning the base pair interval [33,031,935-33,041,244] on the forward strand as indicated in Ensembl ENSG00000142168, and it encodes gene products as is for example shown in Ensembl CCDS33536.

The human SOD2 gene is located on chromosome 6 spanning the base pair interval [160,100,148-160,114,353] on the reverse strand as indicated in Ensembl ENSG00000112096, and it encodes gene products as is for example shown in Ensembl CCDS34564 or CCDS5265.

The human SOD3 gene is located on chromosome 4 spanning the base pair interval [24,797,085-24,802,464] on the forward strand as indicated in Ensembl ENSG00000109610, and it encodes gene products as is for example shown in Ensembl CCDS3430.

The human ANK2 gene is located on chromosome 4 spanning the base pair interval [113,739,265-114,304,896] on the forward strand as indicated in Ensembl ENSG00000145362, and it encodes gene products as is for example shown in Ensembl CCDS3702 or CCDS43261.

The human PRKAA1 (also named AMPK) gene is located on chromosome 5 spanning the base pair interval [40,759,481-40,798,476] on the reverse strand as indicated in Ensembl ENSG00000132356, and it encodes gene products as is for example shown in Ensembl CCDS3932 or CCDS3933. The human FYN gene is located on chromosome 6 spanning the base pair interval [111,981,535-112,194,655] on the reverse strand as indicated in Ensembl ENSG00000010810, and it encodes gene products as is for example shown in Ensembl CCDS5094, CCDS5095 or CCDS5096.

The human SGK1 gene is located on chromosome 6 spanning the base pair interval [134,490,387-134,639,196] on the reverse strand as indicated in Ensembl ENSG00000118515, and it encodes gene products as is for example shown in Ensembl CCDS47476, CCDS47477, CCDS47478 or CCDS5170.

The human DKK1 gene is located on chromosome 10 spanning the base pair interval [54,074,056-54,077,802] on the forward strand as indicated in Ensembl ENSG00000107984, and it encodes gene products as is for example shown in Ensembl CCDS7246.

The human SIRT1 gene is located on chromosome 10 spanning the base pair interval [69,644,427-69,678,147] on the forward strand as indicated in Ensembl ENSG00000096717, and it encodes gene products as is for example shown in Ensembl CCDS44412 or CCDS7273.

The human SIRT2 gene is located on chromosome 19 spanning the base pair interval [39,369,197-39,390,502] on the reverse strand as indicated in Ensembl ENSG00000068903, and it encodes gene products as is for example shown in Ensembl CCDS12523 or CCDS46069.

The human SIRT3 gene is located on chromosome 11 spanning the base pair interval [215,427-236,431] on the reverse strand as indicated in Ensembl ENSG00000142082, and it encodes gene products as is for example shown in Ensembl CCDS7691.

The human UCP1 gene is located on chromosome 4 spanning the base pair interval [141,480,588-141,489,959] on the reverse strand as indicated in Ensembl ENSG00000109424, and it encodes gene products as is for example shown in Ensembl CCDS3753.

The human UCP2 gene is located on chromosome 11 spanning the base pair interval [73,685,716-73,693,889] on the reverse strand as indicated in Ensembl ENSG00000175567, and it encodes gene products as is for example shown in Ensembl CCDS8228.

The human UCP4 (SLC25A27) gene is located on chromosome 6 spanning the base pair interval [46,620,678-46,649,356] on the forward strand as indicated in Ensembl ENSG00000153291, and it encodes gene products as is for example shown in Ensembl CCDS43470.

The human GABARAPL1 gene is located on chromosome 12 spanning the base pair interval [10,365,489-10,375,722] on the forward strand as indicated in Ensembl ENSG00000139112, and it encodes gene products as is for example shown in Ensembl CCDS8620.

The human PRKAB1 gene is located on chromosome 12 spanning the base pair interval [120,105,761-120,119,416] on the forward strand as indicated in Ensembl ENSG00000111725, and it encodes gene products as is for example shown in Ensembl CCDS9191.

The human RYK gene is located on chromosome 3 spanning the base pair interval [133,794,023-133,969,494] on the reverse strand as indicated in Ensembl ENSG00000163785.

The human FoxO3A (FoxO3) gene is located on chromosome 6 spanning the base pair interval [108,881,038-109,005,977] on the forward strand as indicated in Ensembl ENSG00000118689, and it encodes gene products as is for example shown in Ensembl CCDS5068.

The human FoxA1 gene is located on chromosome 14 spanning the base pair interval [38,059,189-38,064,239] on the reverse strand as indicated in Ensembl ENSG00000129514, and it encodes gene products as is for example shown in Ensembl CCDS9665.

The human AKT1 gene is located on chromosome 14 spanning the base pair interval [105,235,689-105,262,080] on the reverse strand as indicated in Ensembl ENSG00000142208, and it encodes gene products as is for example shown in Ensembl CCDS9994.

The human PIK3R2 gene is located on chromosome 19 spanning the base pair interval [18,263,928-18,281,343] on the forward strand as indicated in Ensembl ENSG00000105647, and it encodes gene products as is for example shown in Ensembl CCDS12371.

The human LIG1 gene is located on chromosome 19 spanning the base pair interval [48,618,703-48,673,560] on the reverse strand as indicated in Ensembl ENSG00000105486, and it encodes gene products as is for example shown in Ensembl CCDS12711.

According to the invention, the expression “related biallelic markers from a gene or “a gene related biallelic marker” indicates that the biallelic marker can be localized in an enhancer region, a silencer region, the promoter region, the promoter, an untranslated region (UTR), an intron or an exon of said gene. Such a related biallelic marker can for example be located in a region spanning from 10 kb upstream to 10 kb and downstream of the coding sequence of the gene.

In a preferred embodiment according to the invention, the FoxO-centered network related biallelic marker according to the invention is a GSK-3β related biallelic marker, preferably an SNP.

The GSK-3β related biallelic markers according to the invention are preferably selected in the group comprising the following SNPs: rs3107669, rs7431209, rs7620750, rs17810235, rs11919783, rs9851174, rs4687890, rs2199503, rs334555, rs334558, rs3755557, rs17811013 and rs11925899 (shown in Table 1 herebelow).

In another preferred embodiment according to the invention, the FoxO-centered network related biallelic marker according to the invention is a TCERG1 related biallelic marker, preferably an SNP.

The TCERG1 related biallelic markers according to the invention are preferably selected in the group comprising the following SNPs: rs12519022, rs6889741, rs1978708, rs4705334, rs768232, rs3756502, rs2400220, rs6862473, rs3797301, rs2241697 and rs7731904 (shown in Table 2 herebelow).

In another preferred embodiment according to the invention, the FoxO-centered network related biallelic marker according to the invention is a FoxO1 related biallelic marker, preferably an SNP.

The FoxO1 related biallelic markers according to the invention are preferably selected in the group comprising the following SNPs: rs17446593, rs12865518, rs7981045, rs17446593 (shown in Table 3 herebelow).

In a preferred embodiment according to the invention, the FoxO-centered network related biallelic marker according to the invention is a FZD10 related biallelic marker, preferably an SNP.

The FZD10 related biallelic markers according to the invention are preferably selected in the group comprising the following SNPs: rs7953082, rs10848026, rs3741568, rs7966482, rs2078105 (shown in Table 4 herebelow).

### Prognosing the Age-at-Onset of HD Symptoms

One of the purposes of the present invention is, in particular, the use of at least one of the FoxO-centered network related biallelic marker for prognosing the age-at-onset of symptoms of Huntington's disease in an individual at risk of suffering from said disease. Another purpose of said invention is the use of at least one of the FoxO—centered network related biallelic marker, preferably an SNP, for prognosing the speed at which the disease will progress.

Preferably, said at least one FoxO-centered network related biallelic marker corresponds to or includes a GSK-3β, a TCERG1, a FoxO1 or a FZD10 related biallelic marker.

Most preferably, said at least GSK-3β related biallelic marker is selected from the group consisting of the GSK-3β related biallelic markers shown in table 1 herebelow.

TABLE 1
GSK-3β related biallelic markers

| Biallelic | | | Alternatives |
|---|---|---|---|
| marker No. | Position | in SEQ ID No. | alleles |
| 1 (rs9851174) | 27 | 1 | A/G |
| | | (CCTTCAGTCACACTAGGCTTCTT | |
| | | TCARTTTATTGAATACACAAAGCA | |
| | | TTTTG) | |
| 2 (rs17811013) | 27 | 2 | C/G |
| | | (CACATTTCTCAGCAAAGCTTTCC | |
| | | AACSAAAATTTTTATGATAATCCTT | |
| | | TGTC) | |
| 3 (rs11925899) | 27 | 3 | A/C |
| | | (TCATATAGAATATGAGAATGGGA | |
| | | GGTMCATCTGCTGTTTCCTCACT | |
| | | TGGTGG) | |
| 4 (rs3107669) | 27 | 4 | G/T |
| | | (TTAGTACTTACAGGCTAAATACT | |
| | | TAAKTAACAAGCATAAACCAGGG | |
| | | TTATCC) | |
| 5 (rs7431209) | 27 | 5 | A/G |
| | | (CTTCTATTATTAATGGTTCTACTT | |
| | | TGRTAACCCTTTTATTATTGTGAA | |
| | | GCTT) | |
| 6 (rs7620750) | 27 | 6 | A/G |
| | | (TTGAGAATCACCATGTTCAAGCA | |
| | | TAGRCTTGGCTACTGAGTGAGAA | |
| | | ATACTG) | |
| 7 (rs17810235) | 27 | 7 | C/T |
| | | (ACGGTACAGTTCAGTGGCCGGG | |
| | | GAAGYTGTTTTGGAAGCAGAAAG | |
| | | AAGTGAG) | |
| 8 (rs11919783) | 27 | 8 | A/G |
| | | (CACCAAAAAGTAAACAGTTTTCT | |
| | | CTARATAGTGAGAATACAGGCAA | |
| | | ATTTTT) | |
| 9 (rs4687890) | 27 | 9 | A/G |
| | | (GGCGGCCCTTTGGGGATCCTGT | |
| | | CAGGRGTTTATACTGACCCGCCA | |
| | | TCAATGC) | |
| 10 (rs2199503) | 27 | 10 | A/G |
| | | (GCATAGCCTTTTGGGTGAGGAA | |
| | | GTTGRACTAAGATTTCAGAGCTCT | |
| | | CTTGCT) | |
| 11 (rs334555) | 27 | 11 | C/G |
| | | (CATGTAATTATATCTTATTATTAA | |
| | | AASTCTACCAACTCAAAGCTTCCC | |
| | | CCTT) | |
| 12 (rs334558) | 27 | 12 | A/G |
| | | (ACAAGCCCGCATTCGCCCGGGT | |
| | | CAGGRGCTGCTCTGTGTGAGGA | |
| | | GCGCTGTC) | |
| 13 (rs3755557) | 27 | 13 | A/T |
| | | (AGATGCTGTCTCCAGAAAGCACA | |
| | | TGTWAAAGGACCTATATTTGATC | |
| | | AGATTA) | |

Even more preferably, said GSK-3β related biallelic marker is selected from the group consisting of biallelic markers Nos. 1, 2 or 3.

Most preferably, said at least TCERG1 related biallelic marker is selected from the group consisting of the TCERG1 related biallelic markers shown in table 2 herebelow.

TABLE 2
TCERG1 related biallelic markers

| Biallelic | | | Alternatives |
|---|---|---|---|
| marker No. | Position | in SEQ ID No. | alleles |
| 14 (rs3756502) | 27 | 14 | A/G |
| | | (GCCTTTTTCTTGGCTAATCTTATC | |
| | | ATRTTTAACTAAATGCCATTTTAC | |
| | | TGTC) | |
| 15 (rs7731904) | 27 | 15 | C/T |
| | | (ACAGATCTGTATTTGTATGTTGT | |
| | | GTGYATACAGTCACAGACACACA | |
| | | CATTCA) | |
| 16 (rs1978708) | 27 | 16 | A/G |
| | | (GACCCTATCTTGATTTTGACCTT | |
| | | GTCRGTAAAGCACGGGCTAGCAA | |
| | | AATGCC) | |
| 17 (rs2400220) | 27 | 17 | C/T |
| | | (AACAACCAGAAAGGTGCTAGGTT | |
| | | GCAYATTACACATTTGCACTTTGT | |
| | | AATTT) | |
| 18 (rs2241697) | 27 | 18 | A/G |
| | | (GCCCTGTAATGAGATCGCTAGTT | |
| | | CTCRTATGCTTCAAGGTCTGAAA | |
| | | CAGTGA) | |
| 19 (rs12519022) | 27 | 19 | A/G |
| | | (AGTTAGTACAGACATAGTCCACA | |
| | | TTCRGCTTCTCTTAACGTTAACAT | |
| | | TTATT) | |
| 20 (rs6889741) | 27 | 20 | C/T |
| | | (AACCAAGTCTGATTAGAACTTAC | |
| | | AAGYAATGAGGAAAGATGAAGCT | |
| | | GTAGCG) | |
| 21 (rs4705334) | 27 | 21 | C/T |
| | | (CTGCCTATGCCATGGTAAATAGT | |
| | | TCAYTTATGAAATCTCAGGGCAAC | |
| | | TCTTT) | |
| 22 (rs768232) | 27 | 22 | G/T |
| | | (CGGATTTAAGGTAGGTGCTCAAA | |
| | | AATKGCAGTTATTTTTTTAAAGCC | |
| | | TGATT) | |
| 23 (rs6862473) | 27 | 23 | C/G |
| | | (AACTGGATAGGAAGATTAAGTCC | |
| | | TGGSGTTGTGTAGCCCTGTAGGA | |
| | | TGACTG) | |
| 24 (rs3797301) | 27 | 24 | C/T |
| | | (GTTATCCAGGTTTTGTTCCTTTTT | |
| | | ATYTGCTCTCTTTTTGGTGATGGT | |
| | | TTCT) | |

Preferably, said TCERG1 related biallelic marker is selected from the group consisting of biallelic markers Nos. 14 to 19, even more preferably TCERG1 related biallelic marker is the biallelic markers Nos. 14, 16, 17 or 19.

In a preferred embodiment, a combination of at least one GSK-3β related biallelic marker and at least one TCERG1 related biallelic marker is used for prognosing the age-at-onset of symptoms of Huntington's disease in an individual at risk of suffering from said disease.

In a still preferred embodiment, said combination is selected from the group consisting of related biallelic markers Nos. 3, 14 and 16, related biallelic markers Nos. 3 and 17, and related biallelic markers Nos. 1, 2, 3 and 19.

Most preferably, said at least FoxO1 related biallelic marker is selected from the group consisting of the FoxO1 related biallelic markers shown in table 3 herebelow.

TABLE 3
FoxO1 related biallelic markers

| Biallelic | | | Alternatives |
|---|---|---|---|
| marker No. | Position | in SEQ ID No. | alleles |
| 25 (rs17446593) | 27 | 70 | A/G |
| | | (TTCTTCTAACAGGTGAATGCCTTCTC | |
| | | RGTACAGAGTTCCCTCCTCTCACAGC) | |
| 26 (rs12865518) | 27 | 71 | A/G |
| | | (CCAATCCTTACCTATGGTCTCAAACC | |
| | | RCTATAGACACAAATTTAGTTACACA) | |
| 27 (rs7981045) | 27 | 72 | A/G |
| | | (TTTACAATTCATAAGCAATCATTTATR | |
| | | TAAAAATCTACCTGGTAAGGCATAT) | |
| 28 (rs17446593) | 27 | 73 | A/G |
| | | (TTCTTCTAACAGGTGAATGCCTTCTC | |
| | | RGTACAGAGTTCCCTCCTCTCACAGC) | |

Preferably, said FoxO1 related biallelic marker is selected from the group consisting of biallelic markers Nos. 25 and 26.

Most preferably, said at least FZD10 related biallelic marker is selected from the group consisting of the FZD10 related biallelic markers shown in table 4 herebelow.

TABLE 4
FZD10 related biallelic markers

| Biallelic | | | Alternatives |
|---|---|---|---|
| marker No. | Position | in SEQ ID No. | alleles |
| 29 (rs7953082) | 27 | 74 | A/T |
| | | (TTCTAATAATCCAAGGGTGCATGCAT | |
| | | WAAAAAATGATTATTTGTGCCTAACA) | |
| 30 (rs10848026) | 27 | 75 | C/G |
| | | (ATCCAGTTGCACGAGTTCGCGCCGCT | |
| | | SGTGGAGTACGGCTGCCACGGCCACC) | |
| 31 (rs3741568) | 27 | 76 | A/G |
| | | (AAAAAGCCACAAAATGTAGCACTGAA | |
| | | RGGTTTCTGAGGGCTGGGCCTCTTAG) | |
| 32 (rs7966482) | 27 | 77 | C/G |
| | | (CTGCTGCACCTTTATTCCAAGCAAAA | |
| | | SGGGAACAAGGTTGCATGGCAAAGAG) | |
| 33 (rs2078105) | 27 | 78 | G/T |
| | | (AGGGAAAGACAGAGAATATTTTTCAC | |
| | | KGCTGTAGAGGCTCTGTGCCTGCCTT) | |
| 34 (rs11060759) | 27 | 79 | A/T |
| | | (TCTGTAATGTAAGAAATCAGTTAATTW | |
| | | AAAAAAAATTGCAAAATCTCCATAA) | |

Preferably, said FZD10 related biallelic marker is selected from the group consisting of biallelic markers Nos. 29 to 34, even more preferably FZD10 related biallelic marker is the biallelic markers Nos. 33, 31 and 34.

In a preferred embodiment, a combination of at least one FoxO1 related biallelic marker and at least one FZD10 related biallelic marker is used for prognosing the age-at-onset of symptoms of Huntington's disease in an individual at risk of suffering from said disease.

In a preferred embodiment, said combination is selected from the group consisting of related biallelic markers Nos. 27, 28, 29, 30, 31, 32 and 33.

In another preferred embodiment, a combination of at least one FZD1010 related biallelic marker and at least one GSK-3β related biallelic marker is used for prognosing the age-at-onset of symptoms of Huntington's disease in an individual at risk of suffering from said disease.

In a preferred embodiment, said combination is selected from the group consisting of related biallelic markers Nos. 29, 30, 31, 32, 33, 34 and 3.

In another preferred embodiment a combination of at least one GSK-3β related biallelic marker, at least one TCERG1 related biallelic marker, at least one FoxO1 related biallelic marker and at least one FZD10 related biallelic marker, is used for prognosing the age-at-onset of symptoms of Huntington's disease in an individual at risk of suffering from said disease.

It is within the ability of the skilled person wanting to determine the age-at-onset of HD symptoms to choose the alleles or the combinations of alleles to be used on the basis of well-known statistical methods.

In a further embodiment, the length of CAG repeats in the htt gene is also used in addition to the related biallelic markers according to the invention. The length of the CAG repeat can be assessed by methods known by the skilled in the art and notably, as described in the example, by PCR amplification, using standard conditions, across the CAG repeat or across the combined length of the CAG repeat and the CCG repeat, with correction for the CCG repeat notably as previously described (Passani et al. 2000 Hum Mol Genet. 9:2175-2182).

### Method of Genotyping

The present invention also relates to a method of genotyping comprising the steps of:

a) obtaining an isolated nucleic acid from a biological sample derived from a single individual; and

b) detecting the nucleotide present at one or more FoxO-centered network related biallelic markers, preferably one or more GSK-3β related biallelic markers and/or one or more TCERG1 related biallelic markers and/or FoxO1 related biallelic markers and/or one or more FZD10 related biallelic markers;

c) optionally determining the length of htt gene CAG repeats; and

d) correlating the result of the genotyping steps with the age-at-onset of symptoms of Huntington's disease.

It is preferred that the nucleotide present at one or more of said FoxO-centered network related biallelic markers is detected for both copies of said allelic markers present in said individual's genome. In a preferred embodiment, the nucleotide present at one or more of said FoxO-centered network related allelic markers is detected by a sequencing assay. Preferably, a portion of a sequence comprising the allelic marker is amplified prior to the determination of the identity of the nucleotide. The amplification may preferably be performed by PCR.

The term “genotypinq” a sample or an individual for an allelic marker involves determining the specific allele or the specific nucleotide carried by an individual at/in a given allelic marker.

Any well-known method of genotyping may be used in the frame of the present invention. Such methods include methods such as e.g. conventional dot blot analysis, single-strand conformational polymorphism analysis, denaturing gradient gel electrophoresis, heteroduplex analysis and mismatch cleavage detection. Another method for determining the identity of the nucleotide present at a particular polymorphic site uses a specialized exonuclease-resistant nucleotide derivative as described in U.S. Pat. No. 4,656,127. Oligonucleotide microarrays or solid-phase capturable dideoxynucleotides and mass spectrometry may also be used. Preferred methods involve directly determining the identity of the nucleotide present at an allelic marker site by sequencing assay, enzyme-based mismatch detection assay, or hybridization assay.

In a preferred embodiment, the specific allele carried by an individual is determined by the TaqMan 5′ exonuclease method on an ABI Prism 7700 Sequence Detection System (Applied Biosystems) as notably described in the Examples. In another preferred embodiment according to the invention, the specific allele carried by an individual is determined by the Illumina GoldenGate genotyping assay as notably described in the Examples.

The method of genotyping of the invention further comprises the step of correlating the result of the genotyping steps with the age-at-onset of symptoms of Huntington's disease. This correlation may be easily performed by the skilled in the art on the basis of well known statistical methods.

Preferably, in the method of genotyping according to the invention, the related FoxO-centered network biallelic markers which are used are GSK-3β related biallelic markers and/or TCERG1 related biallelic markers and/or FoxO1 related biallelic markers and/or one or more FZD10 related biallelic markers. Even more preferably, said GSK-3β related biallelic markers are selected from the group of biallelic markers shown in the table set forth in Table 1 (more preferably biallelic markers Nos. 1, 2 and/or 3), said TCERG1 related biallelic markers are selected from the group of biallelic markers shown in the table set forth in Table 2 (more preferably biallelic markers Nos. 14 to 19), said FoxO1 related biallelic markers are selected from the group of biallelic markers shown in the table set forth in Table 3 and said FZD10 related biallelic markers are selected from the group of biallelic markers shown in the table set forth in Table 4.

In a preferred embodiment, the genotyping method of the invention is performed with biallelic markers No:1, 19, 2 and 3 and the presence of:
i. no rare allele at biallelic marker NO: 1 (rs9851174);
ii. no rare allele at biallelic marker NO: 19 (rs12519022);
iii. two rare alleles at biallelic marker NO: 2 (rs17811013); and
iv. two rare alleles at biallelic marker NO: 3 (rs11925899); indicates that said individual is likely to develop symptoms of Huntington's disease at an early age.

In another preferred embodiment, the genotyping method of the invention is performed with biallelic markers No:1, 19, 2 and 3 and the presence of:
i. two rare alleles at biallelic marker NO: 1 (rs9851174);
ii. two rare alleles at biallelic marker NO: 19 (rs12519022);
iii. no rare allele at biallelic marker NO: 2 (rs17811013); and
iv. no rare allele at biallelic marker NO: 3 (rs11925899); indicates that said individual is likely to develop symptoms of Huntington's disease at a late age.

### Kits for Prognosing the Age-at-Onset of Symptoms of Huntington's Disease

One aspect of the invention pertains to a kit comprising:

a) means for detecting the nucleotide present at one or more FoxO-centered network related biallelic markers (e.g. at one or more GSK-3β related biallelic markers and/or at one or more TCERG1 related biallelic markers and/or at one or more FoxO1 related biallelic markers and/or at one or more FZD10 related biallelic markers);

b) instructions for use in the prognosing the age-at-onset of symptoms of Huntington's disease; and, optionally,

c) one or more reagents.

In such a kit, the means preferably allow detecting the nucleotide present at GSK-3β related biallelic markers and/or TCERG1 related biallelic markers and/or FoxO1 related biallelic markers and/or FZD10 related biallelic markers. Even more preferably, said GSK-3β related biallelic markers are selected from the group of biallelic markers shown in the table set forth in Table 1 (more preferably biallelic markers Nos. 1, 2 and/or 3), said TCERG1 related biallelic markers are selected from the group of biallelic markers shown in the table set forth in Table 2 (more preferably biallelic markers Nos. 14 to 19), said FoxO1 related biallelic markers are selected from the group of biallelic markers shown in the table set forth in Table 3 and said FZD10 related biallelic markers are selected from the group of biallelic markers shown in the table set forth in Table 4.

Said means may for example comprise or consist of primers (e.g. a pair or primers for PCR amplification or a microsequence primer) and/or probes (e.g. a fluorescent probe such as a TaqMan probe or a Molecular Beacon probe).

### Treatment or Prevention of HD

The present invention also concerns lithium or a pharmaceutically acceptable salt thereof for use in the treatment or prevention of Huntington's disease, wherein said lithium or salts thereof is for administration at a low dose.

Lithium is commonly used as a mood stabilizing drug. Patients are usually administered a daily dose of 800 to 1200 mg, divided in 2-3 doses. As used herein, a “low dose” refers to a dose that is lower than 800 mg per day. The dose is preferably inferior to 10 mg per day. The dose may for example be comprised between 100 μg-10 mg, 50 μg-5 mg, 100 μg-10 mg, 100 μg-1 mg, or at about 900 μg per day, more preferably at a dose between 200 μg-2 mg.

The lithium or acceptable salts thereof according to the invention can be administered to a patient together with a pharmaceutically acceptable carrier to form a pharmaceutical composition.

In the context of the invention, the term “treating” or “treatment” means either delaying the onset of Huntington's disease or reversing, alleviating, inhibiting the progress of one or more symptoms of Huntington's disease. In particular, the treatment of the disorder may consist in alleviating the motor symptoms, the cognitive symptoms or psychiatric symptoms.

The term “pharmaceutically acceptable salts” refers to salts which retain the biological effectiveness and properties of the lithium and which are not biologically or otherwise undesirable. For a review of pharmaceutically acceptable salts, see Berge, et al. ((1977) J. Pharm. Sd, vol. 66, 1).

According to the invention, the pharmaceutically acceptable lithium salt can for example be lithium carbonate, lithium citrate, lithium sulphate, lithium aspartate or lithium ororate, preferably lithium carbonate.

The expression “pharmaceutically acceptable carrier” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can notably be formulated for an intravenous, intramuscular, oral, sublingual or subcutaneous use.

The treatment may be chronic and can for example consist in the administration of the pharmaceutical composition each day for 1, 2, 3, 4 or 5 months or more as well as for 1, 2, 3, 4 or 5 years or more.

### BRIEF DESCRIPTION OF THE DRAWINGS

FIG. 1: Effects of DAF-16 co-factors in C. elegans. (A) bar-1/β-catenin and ftt-2/14-3-3 null mutations enhanced touch insensitivity at the tail of 1280 nematodes. *P<0.001. (B) Canonical Wnt pathway genes including lin-44, lin-17 and mig-1 affected wild type touch response and were excluded from subsequent analyses. *P<0.001 versus wild-type. (C) Reducing pry-1/Axin and gsk-3 enhanced 128Q neuronal dysfunction. *P<0.01 versus 128Q animals. N>200 in all graphs, with SEM.

FIG. 2: β-catenin and ucp-4 are required for neuroprotection by sir-2.1 in C. elegans. (A) ucp-4 deletion enhanced 128Q neuronal dysfunction. *P<0.01 compared to 128Q animals. (B) Neuroprotection by increased Sir2 dosage (sir-2.1(O/E)) against 128Q toxicity was lost in animals mutant for bar-1 or ucp-4. *P<0.01 versus sir-2.1(O/E) alone. (C) 128Q transgene expression is unchanged in bar-1 and ucp-4 mutants. N>200 in all graphs, with SEM.

FIG. 3: β-catenin, FoxO3a, SIRT1 and UCPs modulate the survival of mutant htt striatal cells from HdhQ111 mice. (A) 109Q/109Q cells were more susceptible to cell death compared to 7Q/7Q cells. *P<0.01. (B) Effects of β-catenin siRNA and BIO (0.5 μM), used alone or combined. *P<0.01 versus untreated, **P<0.05 versus DMSO controls. (C) Effect of FoxO3a siRNA or overexpression (O/E). ***P<0.001 and **P<0.01 versus untreated. (D) Effects of siRNAs against SIRT1, UCP2 and UCP4. *P<0.01 versus untreated. N=4 in all graphs, with SD.

FIG. 4: The survival of mutant htt striatal cells is regulated by SIRT1 and β-catenin together. (A) Mutant htt cells show increased susceptibility to cell death compared to normal htt cells (***P<0.001 compared to 7Q/7Q). β-catenin siRNA alone reduced the survival of mutant htt striatal cells with no effects in normal htt cells (***P<0.001 compared to untreated 109Q/109Q). SIRT1 overexpression enhanced the survival of mutant htt striatal cells with no effects in normal htt cells (**P<0.01 compared to untreated 109Q/109Q). When SIRT1 overexpression was combined with β-catenin siRNA in mutant htt striatal cells, the effect of β-catenin siRNA was suppressed. (*P<0.05 compared to SIRT1 alone, ***P<0.001 compared to untreated 109Q/109Q). n.s., not significant. n=3 in with SD. (B) Representative western blot showing that striatal cells tranfected with the SIRT1 construct (an active variant lacking an internal segment in the N-terminus) have increased SIRT1 levels. n.a., not applicable.

FIG. 5: Characterization of canonical Wnt members and sir-2.1 activity in polyQ nematodes: aggravation of 128Q neuronal dysfunction by sir-2.1 LOF is cell-autonomous. sir-2.1 LOF aggravated neuronal dysfunction in 128Q animals with no effet detected in 19Q animals, and this effect was reversed by overexpressing SIR-2.1 (O/E) fused to a fluorescent reporter in touch receptor neurons. Data are mean±SD as compiled from 2-3 independent arrays per polyQ genotype (3 arrays for 128Q, 2 arrays for 19Q). ***P<0.001.

FIG. 6: The UCP-4 promoter is regulated by DAF-16. (A) DAF-16 binds to the UCP-4 promoter. The 5′ region of UCP-4 has 3 consensus DAF-16 binding sites (upper panel; only 2 sites are shown; Binding site 1 has two closely located consensus sites) that are separated by 3.7 kb. One of the binding sites is located 894 bp upstream of the ATG (binding site 2) while two others are located 4.655 and 4.964 kb upstream of ATG. Since the latter sites are closely situated, primers were designed for the site at 4.655 kb (binding site 1). Primers were also designed for region 654 bp downstream of the stop codon as a control site in the 3′-region. Chromatin immunoprecipitation (lower panel) using anti-DAF-16 antibody (lower panel) using N2, daf-2(e1370), daf-16(mgDf50) and daf-16(mgDf50);daf-2(e1370). The binding was normalized to that of N2. (B) DAF-16 overexpression increases the activity of the UCP-4 promoter in C. elegans. Contructs encoding mCherry under the control of the UCP-4 promoter (1768 bb) were stably expressed in a wild-type N2 strain or strain overexpressing DAF-16::GFP (TJ356), and the intensity of mCherry signals was quantified in late L4 animals in the pharynx area, an area where DAF-16 isoforms and UCP-4 are known to be expressed. Data are mean±SEM as compiled from two independent arrays per genotype and greater than 60 animals/array. ***P<0.005 compared to wild type animals (ANOVA and Tukey multiple comparisons).

FIG. 7: The GSK-3B inhibitor BIO is neuroprotective via FoxO signaling in nematodes. (A) Neuronal dysfunction in 128Q nematodes is higher compared to 190 nematodes (see FIG. 5). BIO rescues expanded polyQ neurotoxicity (*P<0.001 and **P<0.01 versus DMSO controls) with no effect on 19Q controls. BIO rescuing activity was lost in mutants for daf-16, sir-2.1, bar-1 and ucp-4. Dilution factor is 3×. (B) BIO reduced AxSw in PLM cells of 128Q animals CP<0.002 versus DMSO controls).

FIG. 8: BIO does not modify trangene expression in 128Q nematodes.

FIG. 9: Lithium chloride protects C. elegans neurons in a daf-16/FoxO dependent manner. Neuronal dysfunction in 128Q nematodes is higher compared to 19Q nematodes (see FIG. S1B). Lithium chloride rescued expanded polyQ toxicity in C. elegans neurons in a dose-dependent manner with no effects on 19Q controls (*P<0.01, **P<0.05 versus untreated 128Q controls). The rescuing activity of lithium chloride was lost in daf-16 mutants. n>200, with SEM. Dilution factor is 3×.

FIG. 10: 109Q/109Q cells have increased levels of β-catenin. Representative western blot image showing that 109Q/109Q cells had higher levels of β-catenin compared to 7Q/7Q cells (n=3, P<0.05).

FIG. 11: 109Q/109Q cells have similar levels of FoxO3a. Representative western blot image showing that 109Q/109Q cells had higher levels of FoxO3a compared to 7Q/7Q cells.

FIG. 12: Expression analysis for FIG. 3. (A) Mutant htt expression was unchanged by either treatment with BIO or β-catenin siRNA. β-catenin expression is increased by BIO (n=3, P<0.02) and reduced by β-catenin siRNA (n=3, P<0.05). (B) Left: FoxO3a expression was reduced by FoxO3a siRNA (n=3, P<0.05) and increased by FoxO3a O/E (n=3, P<0.05). Right: mutant htt expression was unchanged. (C) Mutant htt expression was unchanged by siRNAs against SIRT1, UCP2 and UCP4. Shown are representative western blots.

FIG. 13: SiRNA efficiency in109Q/109Q cells. Left panel is a representative western blot image demonstrating that 109Q/109Q cells have lower SIRT1 expression levels upon SIRT1 siRNA treatment (P<0.05). Quantitative RT-PCR experiments indicate that SIRT2, SIRT3, UCP2 and UCP4 siRNA decreased mRNA levels of SIRT2, SIRT3, UCP2 and UCP4, respectively (P<0.05). n=5, with SD. Of note, The effect of SIRT2/3 siRNA on target protein expression could not be evaluated, as the antisera were repeatedly unable to detect any protein in western blot experiments. The same applied to the UCP2 and UCP4 antiserum.

FIG. 14: Effect of SIRT1 and β-catenin on UCP4 expression. The expression of the UCP4 gene is upregulated by β-catenin siRNA and dowregulated by SIRT1 overexpression (O/E) in 109Q/109Q cells (n=4 with SD; *P<0.05). No effect was observed in 7Q/7Q cells.

FIG. 15: Effect of SIRT1 and β-catenin on UCP2 expression. The expression of the UCP2 gene is downregulated by β-catenin siRNA with no effect by SIRT1 overexpression (O/E) in 109Q/109Q cells (n=4 with SD; *P<0.05). No effect was observed in 7Q/7Q cells. ns: not significant

FIG. 16: ROS levels in striatal cells. 109Q/109Q striatal cells have higher ROS levels compared to 7Q/7Q cells (n=4 with SD; **P<0.01).

FIG. 17: Effects of β-catenin and SIRT1 on ROS levels in striatal cells. β-catenin siRNA increased ROS levels in mutant htt striatal cells with no effects in normal htt cells, whereas SIRT1 overexpression (O/E) decreased ROS levels in both mutant and normal htt cells (n=4 with SD; *P<0.05 and **P<0.01 compared to untreated). ns: not significant.

### EXAMPLES

### A. Material and Methods

### 1) Nematode Experiments

The wild-type strain of C. elegans used was Bristol N2. Standard methods of culturing and handling worms were used. All strains were scored at 20° C. Touch tests, scoring of PLM cell processes, drug response assays and quantitative Real-Time PCR were performed as previously described (Parker et al. 2005 Nat Genet. 37:349-350). Western analysis was performed using standard protocols and htt fusion proteins detected with the GFP antibody ab6556 (Abcam). Mutations and transgenes used in this study were: aak-2(ok524), bar-1(ga80), cfz-2(ok 1201), cst-1(tm1900), ctl-2(ok1137), cwn-1(ok546), cwn-2(ok895), daf-2(e1370), daf-16(mgD150), dhc-1(or195), dnc-1(or 404), egl-20(n585), ftt-2(n4426), geln3[sir-2.1(+)], igls1[mec-3::htt57Q128::CFP;mec-7::YFP;lin-15(+)], igls245[mec-3::htt57Q19::CFP; mec-7::YFP; lin-15(+)]. lin-17(n671), lin-44(n1792), mig-1(e1787), pop-1(q624), rrf-3(pk1426), sir-2.1(ok434), sod-3(gk235) and ucp-4(ok 195). All strains were obtained from the C. elegans Genetics Center (University of Minnesota, Minneapolis) except for cst-1(tm1900) which was from the National Bioresource Project for the Nematode (Tokyo, Japan). For strain construction with polyQ transgenes, mutants were verified by visible phenotypes, PCR analysis for deletion mutants, sequencing for point mutations or a combination thereof. Deletion mutants were outcrossed a minimum of three times to wild-type and the geln3[sir-2.1(+)] strain outcrossed four times to wild-type prior to use.

Constructs encoding SIR-2.1 constructs were generated as it follows. The cDNA of sir-2.1 was assembled with a bicistronic GFP (biGFP) by PCR fusion. The sir-2.1 cDNA was obtained from wildtype animals by RT-PCR, using RV197 (5′ GGG GAC AAC TTT GTA TAC AAA AGT TGA TGT CAC GTG ATA GTG GCA AC (SEQ ID NO:25)) and RV198 (5′ GTG AAA GTA GGA TGA GAC AGC TCA GAT ACG CAT TTC TTC AC (SEQ ID NO:26)) primers. RV198 contains a sequence complementary to the 5′ region of biGFP. biGFP was amplified from pAN51 using RV192 (5′ GCT GTC TCA TCC TAC TTT CAC (SEQ ID NO:27)) and RV178 (5′ GGG GAC CAC TTT GTA CAA GAA AGC TGG GTA TTA TAG TTC ATC CAT GCC ATG TGT A (SEQ ID NO:28)). Then, both PCR products were fused by nested PCR using primers RV197 and RV178. These primers contain respectively attB5 and attB2 sequences for recombination in the pDONR221-P5-P2 vector, using the Gateway system (Invitrogene). In parallel, a clone, in pDONR221-P1-P5r, was produced containing the promoter of mec-3, mec-3p, using primers RV3 (5′ GGG GAC AAG TTT GTA CAA AAA AGC AGG CTC CTG CAG GTA CCC GGA GTA GTT G (SEQ ID NO:29)) and RV4 (5′ GGG GAC AAC TTT TGT ATA CAA AGT TGT GGC GCG CCA ATG CGC GAA ATT GTG GCT ACT C (SEQ ID NO:30)). Both clones were used to assemble mec-3p and sir-2.1-biGFP, using Gateway technology, in the destination vector pDEST-AN, which is suitable for C. elegans transgenesis. Constructs encoding mCherry under the control of the UCP-4 promoter (1768 bp) were generated as it follows. The promoter of UCP-4 (from position 1768 bp upstream of the ATG of ucp-4) was fused to mCherry::unc-54 Terminator. Primers used to amplify ucp-4 were Forward 5′-TTTTGCGTTTGCTCGTCGCAC (SEQ ID NO:31) and Reverse 5′-AGTCGACCTGCAGGCATGCAAGCT (SEQ ID NO:32). mCherry was amplified from a plasmid generated by replacing GFP from pPD95.75 by mCherry and by using the primers Forward 5′-AGCTTGCATGCCTGCAGGTCGACT (SEQ ID NO:33) and Reverse 5′GGAAACAGTTATGTTTGGTATATTGGG (SEQ ID NO:34). All of these constructs were verified for sequence integrity.

For transgenesis, 20 ng/μl of DNA of the constructs were injected into 19Q and 128Q nematodes, together with pPD118.33 (a plasmid containing myo-2p::GFP), at a final total DNA concentration of 100-150 ng/μl, using standard methods. At least two independent strains from each construct were isolated to perform the touch tests. Strains stably expressing the mCherry reporter were crossed to the wild-type N2 strain or DAF-16::GFP over-expressing strain TJ356 (insertion zls356IV), and animals homozygous for each allele (wt/wt, daf-16::GFP/daf-16::GFP) were isolated. The mCherry signals were scored using a Zeiss fluorescence microscope (10×) and quantification was performed using ImageJ.

Touch tests involved scoring for the response to light touch at the tail by using a fine hair. Touch test were performed by scoring 10 touches at the tail of the animal for a minimum of 200 animals per genotype. Ordinarily, wild-type animals will respond by backing away from the touch. The responses were recorded for every animal such that, for example, 3 responses out of 10 at the tail is given as 30% responsiveness, and the mean values for responsiveness were retained for comparison of nematode groups.

Chromatin immunoprecipitation (ChIP) was essentially performed as described previously (Oh et al. 2006 Nat Genet. 38:251-57, Mukhopadhyay et al. 2008 Nat Protoc 3:698-709). wild type (N2), daf-2(e1370), daf-16(mgDf50) and daf-16(mgDf50); daf-2(e1370) worms were grown in liquid (S-basal medium supplemented with 50 mg/L and Nystatin and 50 mg/L Streptomycin) at 20° C. and were fed everyday with 1 ml of concentrated HB101 bacteria (single colony grown overnight in 1 L LB medium, bacterial pellet resuspended in 25 ml 1×M9 buffer) to obtain a mixed stage culture. A worm pellet of approximately 250 μl was then resuspended in 3 ml formaldehyde crosslinking buffer (CLB; 1% formaldehyde in 1×PBS) and transferred to a glass homogenizer. Abrasions were created in the worm cuticle by plunging the glass plunger 8 times. The worm suspension was then collected in a fresh tube and the homogenizer rinsed with an additional 1 ml of CLB; resulting in a total of a 4 ml suspension which was then incubated for 20 minutes on a shaker at room temperature (Crosslinking). This was followed by quenching using 200 μl of 2.5 mM glycine. The crosslinked worms were washed three times with 1×PBS and snap frozen in liquid nitrogen. The frozen pellet was then resuspended in 3 ml HEPES lysis buffer [50 mM HEPES-KOH, pH 7.5, 150 mM NaCl, 1 mM EDTA, 0.1% (wt/vol) sodium deoxycholate, 1% (vol/vol) Triton X-100, 0.1% (wt/vol) SDS, 1 mM PMSF, diluted proteinase inhibitor cocktail (10 μl/ml; Sigma)], and split into 750 ml aliquots. Each of the aliquots were sonicated 8 times using a Misonix sonicator 3000 (Misonix Inc., New York, N.Y.) with output setting 8, power output of 30W for 10 sec. The suspension was then centrifuged at high speed at 4° C. and supernatant collected. Supernatant equivalent to 2 mg protein was pre-cleared using 50 μl of prewashed salmon sperm DNA/protein-A agarose beads (Upstate, USA) and was used for immunoprecipitation with anti-DAF-16 antibody (25 μl) overnight at 4° C. About 5% of the precleared supernatant was used as Input or WCE to isolate genomic DNA. The antibody-protein-DNA complex was then sequestered using 50 μl of prewashed salmon sperm DNA/protein-A agarose beads (1.5 hr at 4° C.). The complex was washed twice using Wash buffer 1 [50 mM HEPES-KOH, pH 7.5, 150 mM NaCl, 1 mM EDTA pH 8.0, 1% (wt/vol) Sodium deoxycholate, 1% (vol/vol) Triton X-100, 0.1% (wt/vol) SDS, 1 mM PMSF], twice with Wash buffer 2 [50 mM HEPES-KOH, pH 7.5, 1 M NaCl, 1 mM EDTA, pH 8.0, 0.1% (wt/vol) Sodium deoxycholate, 1% (vol/vol) Triton X-100, 0.1(wt/vol) % SDS, 1 mM PMSF], once with Wash buffer 3 [50 mM Tris-Cl, pH 8.0, 0.25 mM LiCl, 1 mM EDTA, 0.5% (vol/vol) NP-40, 0.5% (wt/vol) Sodium deoxycholate] and three times with 1×TE [10 mM Tris-Cl, pH 8.0, 1 mM EDTA]. The beads were then resuspended in 250 μl Proteinase K buffer [50 mM Tris-Cl, pH 8.0, 25 mM EDTA, pH 8.0, 1.25% (wt/vol) SDS] containing 2 μl Proteinase K (20 mg/ml) and incubated at 42° C. for 2 h. The input was resuspended in 250 μl Proteinase K buffer containing 5 μl Proteinase K (20 mg/ml) and incubated at 55° C. for 5 h. All samples were reverse crosslinked at 65° C. overnight and DNA was recovered following phenol-chloroform extraction and ethanol precipitation. ChIP samples were resuspended in 40 μl of 10 mM Tris-Cl, pH 8.0 while inputs were resuspended in 200 μl. Binding was determined by real-time PCR using the following primers: binding site 1-tctgtgtcagcagcttttcg (SEQ ID NO:35) and acggccgtccgtaatagata (SEQ ID NO:36), binding site 2-tgagcaagttgtaatggggtta (SEQ ID NO:37) and catctggcacttatggggtta (SEQ ID NO:38), 3′-region-aaccaggagcaccattcaac (SEQ ID NO:39) and gcaacttcttcctgctgacc (SEQ ID NO:40).

### 2) Mammalian Experiments

Cell death assays for HdhQ111 mouse striatal cells expressing full-length htt with either wild-type (7Q/7Q) or mutant (109Q/109Q) polyglutamines were performed as previously described (Arango et al. 2006 Neurosci 26:4649-59). jetPEI was used for transfection with cDNA and jetSI-ENDO for siRNA assays as indicated by the manufacturer (PolyPlus Transfection). siRNA oligos were obtained from Clontech (β-catenin) and QIAGEN (SIRT1, SIRT2, SIRT3, UCP2 and UCP4). Mixes of 3-4 different siRNA sequences per gene (25 or 33 nM) were systematically tested for modulation of cell survival and siRNA target gene expression, followed by the evaluation of individual siRNA sequences at optimal concentration (25-100 nM). Effects on cell survival were considered to be reliable if 2 different siRNAs showed similar effects on target mRNA/protein expression and cell survival. The siRNA sequences shown in figures are as it follows: β-catenin siRNA, 5′-GGCTTTTCCCAGTCCTTCATT-3′ (100 nM) (SEQ ID NO:41); FoxO3a siRNA 5′-TTCTGAACGCGCATGAAGCTT-3′ (SEQ ID NO:42) (100 nM); SIRT1 siRNA, 5′-GATTGTTATTAATATCCTTTT-3′ (SEQ ID NO:43) (25 nM); SIRT2 siRNA 5′-TTGGGTGAAGTTCTATTTGTT-3′ (SEQ ID NO:44) (25 nM); SIRT3 siRNA, 5′-TTACCGATCAACATGCTAGTT-3′ (SEQ ID NO:45) (25 nM); UCP2 siRNA, 5′-TAAAGGTGTCTCGTTCTTCTT-3′ (SEQ ID NO:46) (33 nM), and UCP4 siRNA sequences 5′-GCCTATAGTATGTGCTTAATT-3′ (SEQ ID NO:47) (33 nM). The constructs encoding an active variant of SIRT1 (2 kb cDNA) lacking an internal segment in the N-terminus and full length FoxO3a were pcDNA3.1-sirt1-Flag and pcDNA3.1-FoxO3a-HA, respectively. Cells were subjected to DAPI staining and cell death was scored 48 hours after cell transfection by counting picnotic versus normal nuclei in DAPI- and JetSI-ENDO-positive cells. Cytoplasmic and nuclear proteins were extracted as previously described (Arango et al. 2006 Neurosci 26:4649-59), separated by SDS-PAGE and analyzed by Western blotting using the following primary antibodies: Mouse anti-SIRT1 (Upstate, 1:1000), mouse anti-SIRT2 (Santa Cruz, 1:100), rabbit anti-SIRT-3 (Abgent, 1:100), rabbit-anti-β-catenin (Cell Signaling, 1:5000), mouse anti-FoxO3a (Cell Signaling, 1:1000), goat-anti-UCP2 (Santa Cruz, dilution), rabbit-anti-UCP4 (Abcam, 1:25), mouse anti-actin (Invitrogen, 1:2000), anti-htt (4C8, Chemicon, 1:5000). Secondary antibodies were goat-anti-mouse IgG HRP-conjugated and goat-anti-rabbit IgG HRP-conjugated (Biorad). Proteins were detected using ECL+ and evaluated by densitometry. Quantitative Real-Time PCR experiments were performed as described in the Supporting Information/Materials and Methods. Chemicals were either purchased from Sigma (Resveratrol) and Calbiochem (B10, LiCl).

RNA Isolation and qRT-PCR in Striatal Cells.

Quantitative RT-PCR was performed on RNA extracted from 109Q/109Q mouse striatal cells, transfected with the appropriate siRNAs, to assess gene silencing efficiency. Two days after cell transfection, RNA was extracted with a Qiagen Rneasy kit and Dnasel (Sigma) treatment (as per the manufacturers protocol). Single strand cDNA synthesis was done using oligoDT, random hexamer priming and 100 ng of total RNA with Absolute 2-Step QRT-PCR SYBR ROX kit (AbGene). Quantitative PCR was performed using SYBR® Green with the ABI PRISM® 7700 Sequence Detection System (Applied Biosystems) and oligonucleotides as it follows: 5′-3′, ucp2-f (TCCTGCTACCTCCCAGAAGA) (SEQ ID NO:48), ucp2-r (TGAGACCTCAAAGCAGCCTC) (SEQ ID NO:49), ucp4-f (GTGACGCCCGCCATTTACAG) (SEQ ID NO:50), ucp4-r (CATCCCTCCAATGACCGATTTCC) (SEQ ID NO:51), sirt2-f (CCCTTCGCCTCCCTCATC) (SEQ ID NO:52), sirt2-r (GTCCCTGTAAGCCTTCTTGG) (SEQ ID NO:53), sirt3-f (TGGCTGACTTCGCTTTGG) (SEQ ID NO:54), sirt3-r (TCCACACCATGAACTACATCC) (SEQ ID NO:55), rpl13a-f (CTGCTGCTCTCAAGGTTGTTC) (SEQ ID NO:56), rpl13a-r (CTGCTTCTTCTTCCGATAGTGC) (SEQ ID NO:57), hprt-f (TTTGCCGCGAGCCG) (SEQ ID NO:58), hprt-r (TAACCTGGTTCATCATCGCTAATC (SEQ ID NO:59)). Assays and data analysis were performed according to the manufacturer's protocol (User Bulletin #2, ABI PRISM® 7700 Sequence Detection System, Perkin Elmer). All samples were run at least in triplicate using rpl13a or hprt as the calibrator gene with a dilution of 1/100 of cDNA. The amount of target, normalized to an endogenous reference (ucp2 or ucp4) and relative to the calibrator (rpl13a or hprt) was calculated using the 2^{-ΔΔCT} method and statistical significance determined using paired t tests.

Polymorphism Analysis in DNA Samples from French HD Patients.

Polymorphism analysis for the GSK-3β, FoxO3a and UCP2 genes was performed in 342 HD subjects recruited in the framework of the program “Predictive biomarkers of Huntington's disease” through the French Speaking Huntington's Disease Group. This program was approved by the ethical committee at the Henri Mondor Hospital (Créteil, Paris). Ages at onset were based on the clinical assessment of the examiner as proposed in the Unified Huntington's Disease Rating Scale (Unified Huntington's Disease Rating Scale: reliability and consistency 1996 Huntington Study Group. Mov Disord 11:136-142). Onset could reflect characteristic choreic movements and cognitive and psychiatric symptoms. Onset date was ascertained by interview of the patient and family members, by review of medical records and in some instances by clinical observation of onset in an individual previously examined and found to be asymptomatic. After obtaining informed consent, blood was drawn from HD subjects and DNA extracted. HD was genetically confirmed (CAG repeats>35). The repeat region of CA150 was amplified as follows: the reaction mixture included 500 nM each of forward primer 5′-AACTGACACCTATGCTTG-3′ (SEQ ID NO:60) (fluorescently tagged) and reverse primer 5′-GTTGAAGTGGATACTGCA-3′ (SEQ ID NO:61), approximately 500 ng DNA, buffer G (Epicenter Technologies), and 2.5 U Taq polymerase (Life Technologies). After a 5 min denaturation at 95°, PCR was performed over 33 cycles (95° C.×45 sec, 51° C.×45 sec, 72° C.×1 min), with a 7 min final extension at 72° C. The CAG repeat length in the htt gene was determined by PCR amplification, using standard conditions, across the CAG repeat or across the combined length of the CAG repeat and the CCG repeat, with correction for the CCG repeat as previously described (Passani et al. 2000 Hum Mol Genet. 9:2175-2182). Single nucleotide polymorphisms (SNPs) were tested in the GSK-3β (1 SNP in the promoter, 1 intronic SNP), FoxO3a (1 SNP in the promoter, 1 intronic SNP), and UCP2 (1 SNP in the promoter) genes. All SNPs were selected based on their previously-described associations to human pathology. The SNPs in the promoter (rs334558) and intronic region (rs6438552) of the GSK-3β gene (Kwok et al. 2005 Ann Neurol 58:829-839) were analyzed by the TaqMan 5′ exonuclease method on an ABI Prism 7700 Sequence Detection System (Applied Biosystems) using the TaqMan® predesigned SNP Genotyping Assays reagents from Applied Biosystems, Inc. (Foster City, Calif.) according to the manufacturer's protocol (Bulletin: TaqMan® SNP Genotyping Assays, Applied Biosystems). The SNP in the promoter region of the FoxO3a gene (Kim et al. 2006 Obesity 14:188-193) (c.-343-1582C>T) was analyzed by the TaqMan 5′ exonuclease method on an ABI Prism 7700 Sequence Detection System (Applied Biosystems) using the ABsolute QPCR ROX Mix from AbGene and primers 5′-AGCATCTTATCTCGGGTGTTACCT (SEQ ID NO:62) (forward), 5′-CCAGCCCCGGTGTCC (SEQ ID NO:63) (reverse), 5′-CTGTTAGAACGCGACACC (SEQ ID NO:64) (probe-1(VIC)) and 5′-CTGTTAGAACACGACACC (SEQ ID NO:65) (probe-2(FAM)) for c.-343-1582C>T. The SNPs in the intronic region of FoxO3a (Kuningas et al. 2007 Eur J Hum Genet. 15:294-301) (rs13220810) and at position −866 in the promoter region of the UCP2 gene (Esterbauer et al. 2001 Nat Genet. 28:178-83) (G-866A) were examined by PCR-RFLP. Primers for rs13220810 were 5′-ACACGCCACAGATACATAGC (SEQ ID NO:66) (forward) and 5′-AGTCAACCTCCTCCAATCC (SEQ ID NO:67) (reverse). PCR conditions were an initial denaturation at 95° C. for 5 min and 34 cycles (95° C.×30 sec, 55° C.×30 sec, and 72° C.×30 sec), with a final extension for 7 min at 72° C. Amplification yields a 186-bp fragment that contained one TspRl restriction site for the C-allele and none for the T-allele. Primers for G-866A were 5′-CACGCTGCTTCTGCCAGGAC (SEQ ID NO:68) (forward) and 5′-AGGCGTCAGGAGATGGACCG (SEQ ID NO:69) (reverse). PCR conditions were an initial denaturation at 95° C. for 5 min and 34 cycles (95° C.×30 sec, 67° C.×30 sec, and 72° C.×30 sec), with a final extension for 7 min at 72° C. Amplification yields a 360-bp fragment that contained one Mlul restriction site for the G-allele and none for the A-allele. Following enzymatic digestion, PCR products were resolved on 2% agarose gel electrophoresis and visualized by ethidium bromide staining. Statistical testing was performed by using SAS 9.1 (SAS Institute Inc., Cary, N.C.) and SPSS 12.0 (SPSS Inc., Chicago, Ill.). To test for the combined effect of the TCERG1 and GSK-3β genes (Table 5), these two genes were added as one grouped independent variable to adjust the statistical power of the analysis with the small number of markers tested. Adjusted R² values were computed for all of the analyzes.

Analysis of GSK-313, TCERG1, FoxO1 and FZD10 Polymorphism in DNA Samples from the European HD Cohort (not Including French Patients).

Polymorphism analysis for the GSK-3β and TCERG1 genes was performed in 538 samples from study sites in European countries within the European Huntington's Disease Network (EHDN, http://www.euro-hd.net). Participants gave informed written consent according to the International Conference on Harmonisation-Good Clinical Practice (ICH-GCP) guidelines (http://www.ich.org/LOB/media/MEDIA482.pdf). For participants who lacked capacity to consent study sites adhered to country-specific guidelines for obtaining consent. Ethical approval was obtained from the local ethics committee for each study site contributing to REGISTRY. Data was collected using electronic case report forms available in the country's native languages. All participants were assigned a 9-digit pseudonym created using a secure one-way hash algorithm. No identifying data were stored on the EHDN server. At each centre, clinicians with long standing experience in HD took a careful history and examined patients clinically; AAO, symptom-at-onset, and clinical signs were scored using the Unified Huntington's Disease Rating Scale (UHDRS)(1996 Mov Disorder 11:136-142). Blood was collected and shipped to BioRep at room temperature for genetic analysis and lymphoblastoid cell line creation (BioRep, Milan, Italy)(Beck et al. 2001 Cancer Epidemiol Biomarkers Prey 10:551-554, Bernacki et al 2003 J Mol Diagn 5:227-230). DNA was extracted (Miller et al. 1988 Nucleic Acids Res 16:1215), and htt gene CAG repeat length was analyzed using PCR amplification followed by capillary electrophoresis and the MegaBace Fragment Profiler Software from General Electric (Buckinghamshire, UK)(Warner et al 1993 Mol Cell Probes 7:235-239, Riess et al. 1993 Hum Mol Genet. 2:637). DNA and urine were stored at −80° C. The selection and evaluation of tag SNPs in the GSK-3β and TCERG1 genes were selected using the tagger algorithm as implemented in Haploview v. 1.4 (Barett et al. 2005 Bioinformatics 21:263-265) (Integragen SA, Evry, France). The tagger algorithm was applied to genotyped data from the International HapMap Project (CEU HapMap phase II data). The gene position was extended by 10 kb downstream and upstream. A pairwise tagging of all SNPs with a minor allele frequency above 0.1 was done with a R² of 1.0. To reduce the risk of genotyping failure, all SNPs covered by the gene (+/−10 kb) were submitted to Illumina's Assay Design Tool (GoldenGate) in order to get a per SNP “Design score” roughly corresponding to the likelihood of genotyping success. For all tag SNPs, it was looked for the best surrogate SNP in its bin (the set of SNPs tagged by the tag SNP) defined as the SNP with the best Design score. All SNPs from the bin and the tag SNP can be considered as equivalent from an information point of view since R²=1. This selection procedure resulted in the selection of 13 SNPs for GSK-3β including rs3107669, rs7431209, rs7620750, rs17810235, rs11919783, rs9851174, rs4687890, rs2199503, rs334555, rs334558, rs3755557, rs17811013 and rs11925899 and 11 SNPs for TCERG1 including rs12519022, rs6889741, rs1978708, rs4705334, rs768232, rs3756502, rs2400220, rs6862473, rs3797301, rs2241697 and rs7731904. Single nucleotide polymorphism was analyzed by using the Illumina GoldenGate genotyping assay as previously described (Fan et al. 2006 Nat Rev Genet. 7:632-44) and the Illumina® platform (Integragen SA, Evry, France). Statistical analysis was performed using the R-package Version 2.10.0 (R Development Core Team (2009): ‘R: A language and environment for statistical computing. R Foundation for Statistical Computing, Vienna, Austria. ISBN 3-900051-07-0; http://www.R-project.org). In addition to the complete dataset of European HD patients (528 patients without missing values), two subsets were analyzed: Those patients with size of the larger htt allele between 38 and 53 (508 patients) and those with motor onset only (272 patients). Four linear models were built for each of these sets using In(AAO) as endpoint: First of all, the model using only the larger htt allele was constructed as a reference, then the larger htt allele plus the SNPs from GSK-3β, FoxO1 and FZD10 were considered, then the larger htt allele plus the SNPs from TCERG1 and finally the larger htt allele plus SNPs from GSK-36 and TCERG1, or FoxO1 and FZD10, or FZD10 and GSK-36. All SNPs were coded following an additive inheritance model, the homozygote in the wild-type allele was coded as 0, the heterozygote was coded as 1 and the homozygote on the mutant allele was coded as 2. To arrive at a parsimonious model, forward selection was used utilizing Akaike's information criterion as basis for selection. In addition to the SNPs themselves, all two-way interactions were considered in the model selection process. Adjusted R² values and differences in R² as compared to the model containing htt only were computed for all final models to aid judgement of the additional benefit from incorporating genotypes. Significances of the final models were assessed based on a likelihood ratio test comparing the final model to the respective model containing htt only using an a-level of 0.05.

Statistics.

Statistics of nematode data were performed using one-way ANOVA, with correction for multiple testing by Tukey's Multiple Comparison Test. Data were expressed as mean±SEM for >200 nematodes in each group. Student's t-tests were used for striatal cell data. Data were expressed as mean±SD for >150 cells in each group. Student's t-test were used for gene and protein expression data. All experiments were repeated at least three times. P<0.05 was considered significant. Linear models were utilized for analysis of genotypes in the French and European cohort as detailed above.

### B. Results

### Example 1

### β-catenin and ucp-4 are Required for Neuroprotection by sir-2.1 in C. elegans

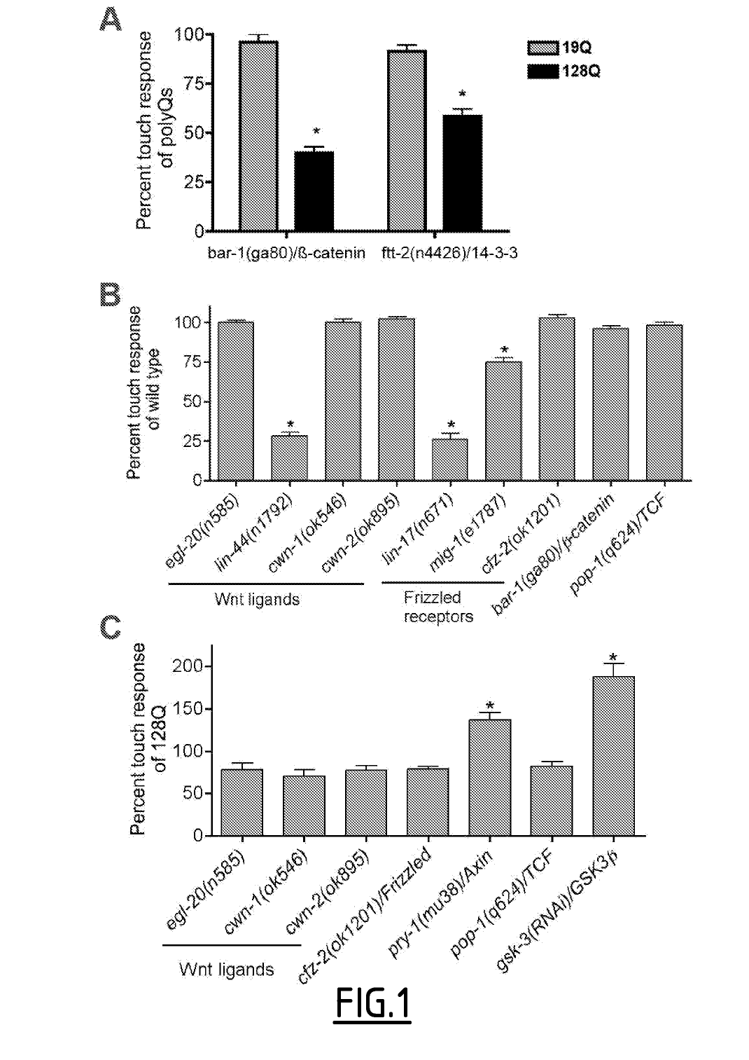

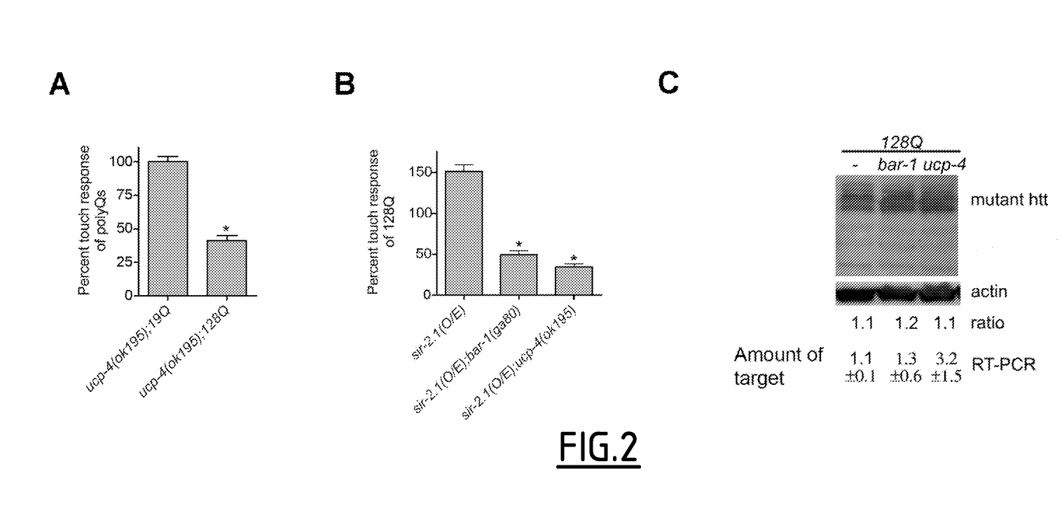

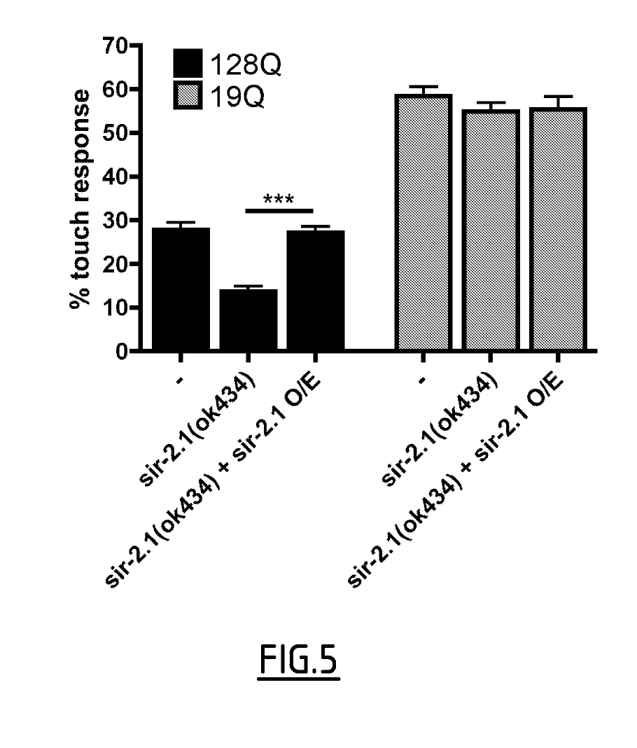

To explore whether protection of expanded-polyQ nematode neurons by the Sir2-FoxO-centered network may involve FoxO co-factors, BAR-1/β-catenin, a major effector of Wnt signaling that binds DAF-16/FoxO, and FTT-2/14-3-3, a major regulator of DAF-16 transcriptional activity were tested. Nematodes expressing normal polyQs in touch receptor neurons show a moderate loss of response touch, whereas expanded-polyQ nematodes show a strong loss of response to touch (85%), a phenotype accompanied by axonal swelling (Parket et al. 2005 Nat Genet. 37:349-50). Loss-of-function mutants for bar-1 and ftt-2 enhanced loss of response to touch in expanded-polyQ nematodes (FIG. 1A), indicating that they are normally protective. Consistent with previous reports, neuron polarity defects were observed in mutants of some of the upstream Wnt pathway components. The remaining Wnt pathway genes did not modify touch response in wild-type (FIG. 1B) or expanded-polyQ (FIG. 1C) animals. Although this suggests that upstream Wnt components are dispensable to protection from expanded polyQs, several Wnt ligands and receptors were expressed in touch receptor cells as shown by microarray analysis upon cell sorting (C. Tourette and coll., submitted elsewhere), which may explain the lack of effect in single mutant analysis. In the canonical Wnt pathway, gsk-3/GSK-3B and pry-1/Axin negatively regulate β-catenin, suggesting their loss may be neuroprotective. Consistently, reducing gsk-3/GSK-3β and pry-1/Axin reversed expanded polyQ toxicity (FIG. 1C). Next, the role of daf-16 downstream effectors was investigated with a focus on mitochondrial uncoupling proteins (UCPs) since they are thought to be essential to neuron survival and since mitochondrial dysfunction is central to neurodegenerative pathogenesis. C. elegans has one UCP gene, ucp-4, and it was hypothesized it may act downtream to DAF-16. The ucp-4 deletion enhanced neuronal dysfunction in 1280 animals (FIG. 2A). Mutations of TOB11.4, a nucleotide transporter related to ucp-4, and clk-1, a conserved enzyme required for ubiquinone biosynthesis did not affect 1280 toxicity, suggesting the effect of ucp-4 is not due to a general impairment of mitochondrial function. Finally, in addition to requiring daf-16, neuroprotection by increased sir-2.1 dosage, a cell autonomous process (FIG. 5), required bar-1 and ucp-4 (FIG. 2B) with no change in transgene expression (FIG. 2C).

### Example 2

### The ucp-4 Promoter is Regulated by DAF-16

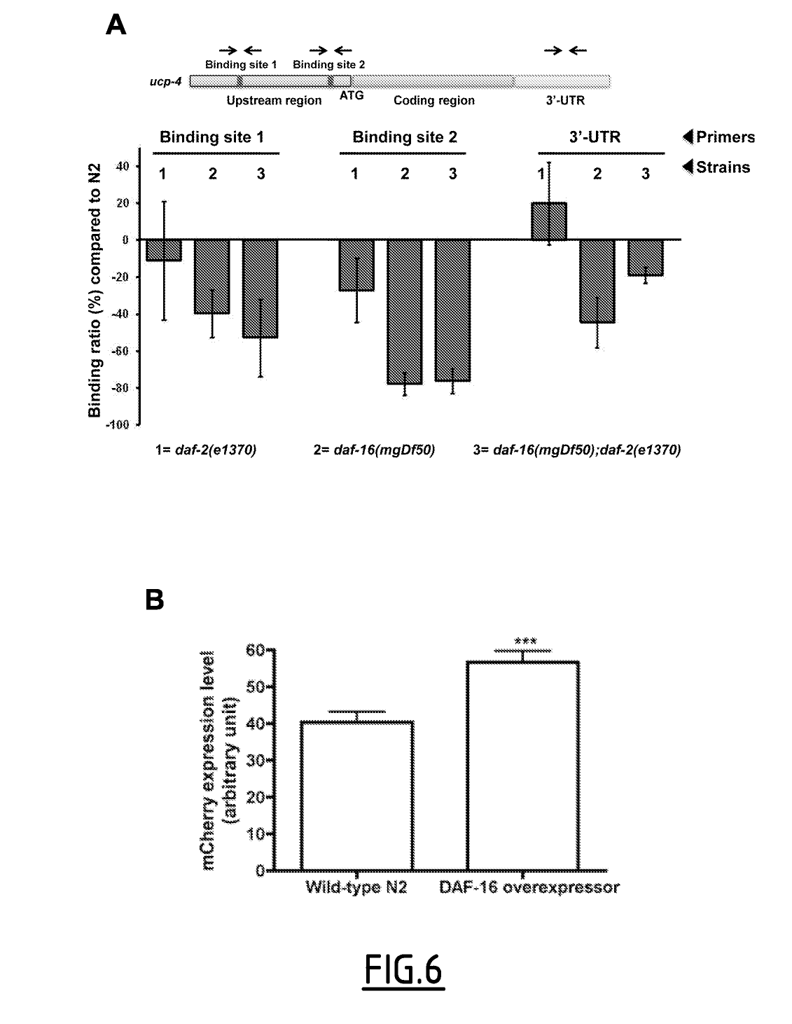

It was then tested whether UCP-4 may regulated by DAF-16. The promoter of ucp-4 was scanned and 3 consensus DAF-16 binding sites (BS) were identified within 5 kb upstream of the translation start site (FIG. 6A). Chromatin Immunoprecipitation (ChIP) showed little difference in binding of DAF-16 to BS in the N2 and daf-2 backgrounds, which may signify daf-2 independent recruitment (FIG. 6A). In contrast, there was dramatic decrease in binding at BS2 when DAF-16 was absent, signifying DAF-16 dependence (FIG. 6A). Some binding of DAF-16 in the 3′-region was observed, which may be due to the fact that the DNA could not be sheared to that extent (FIG. 6A). Additionally, the expression of a mCherry reporter driven by the UCP-4 promoter (1768-bp fragment) was increased in animals bearing DAF-16 overexpression compared to wild-type nematodes (FIG. 6B). Together, these experiments suggested that the UCP-4 promoter is regulated by DAF-16.

### Example 3

### GSK-3β Inhibitors Require bar-1, sir-2.1, daf-16 and ucp-4 for Neuroprotection

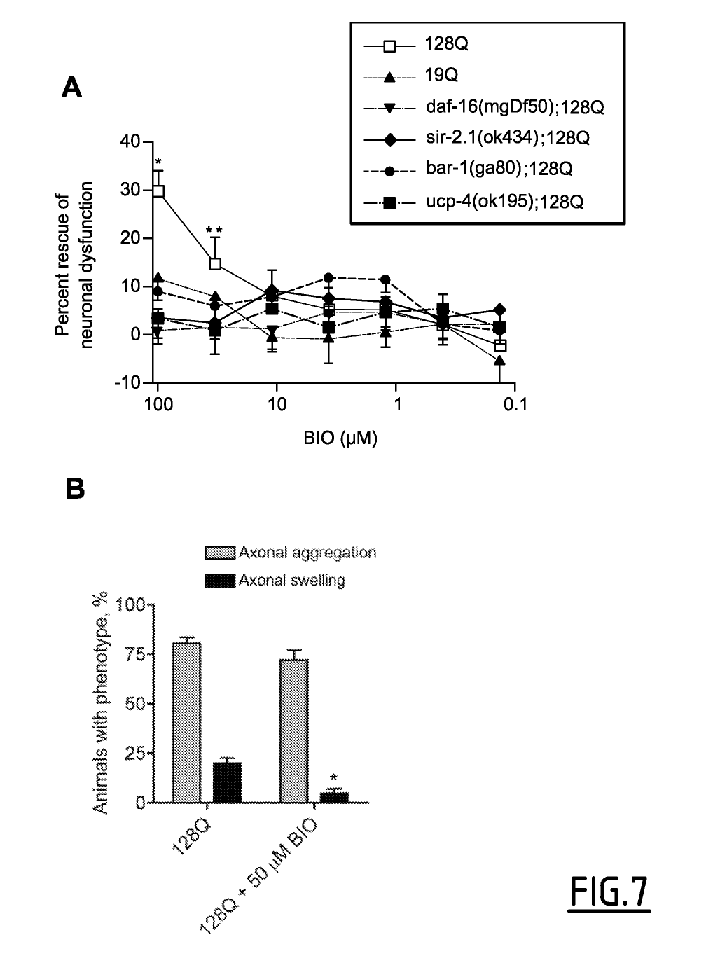

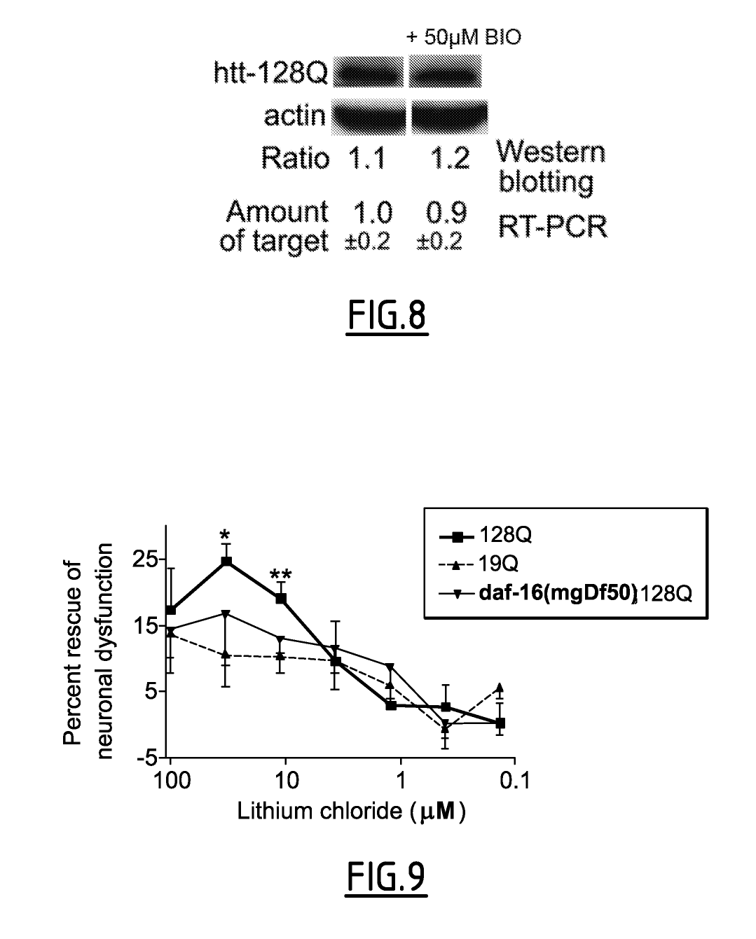

Since β-catenin is required for neuroprotection by sir-2.1 (FIG. 2), neuroprotection by inhibitors of β-catenin degradation was examined. 6-bromoindirubin-3′-oxime (B10), a selective inhibitor of GSK-3β, reduced neuronal dysfunction in 1280 animals in a bar-1 dependent manner (FIG. 7A) without changing transgene expression (FIG. 8). Consistent with the interaction of sir-2.1 with bar-1 and ucp-4 (FIG. 2), BIO had no effect in mutants for sir-2.1, daf-16 and ucp-4 (FIG. 7A). BIO reduced axonal swelling (FIG. 7B), which may reflect increased neuronal health. Lithium chloride, a drug that inhibits GSK-3β and protects against expanded polyQs, protected from 128Q neuronal dysfunction and required daf-16 activity (FIG. 9).

### Example 4

### β-catenin, FoxO3a, SIRT1 and UCPs Modulate the Survival of Mouse Striatal Cells

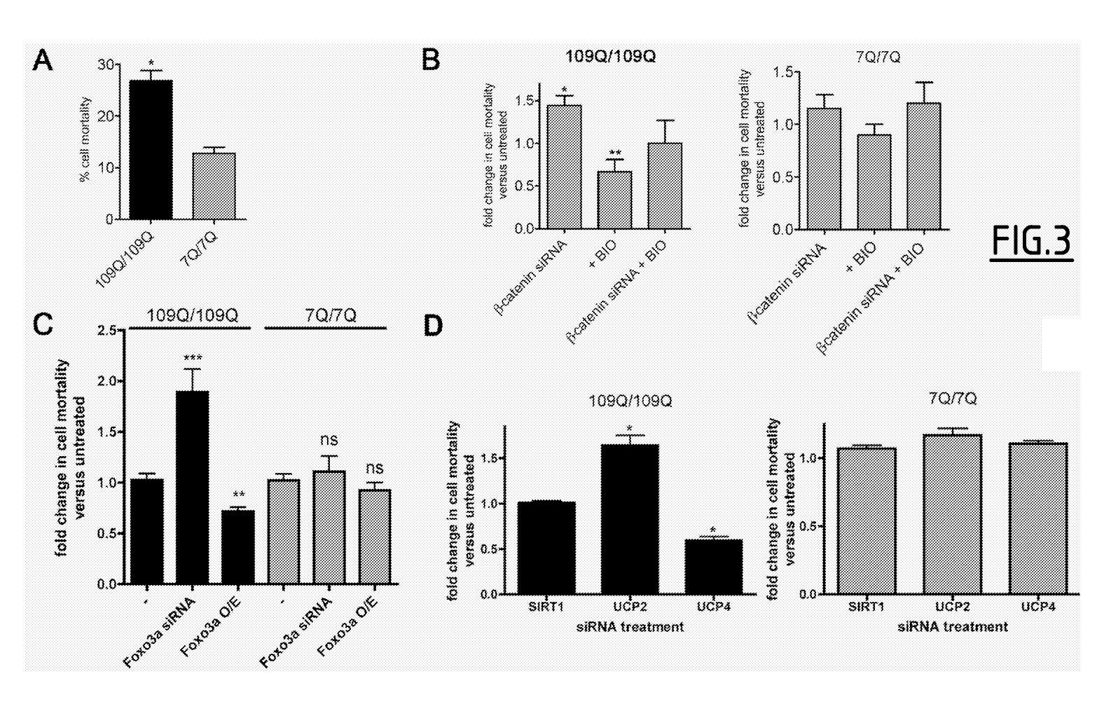

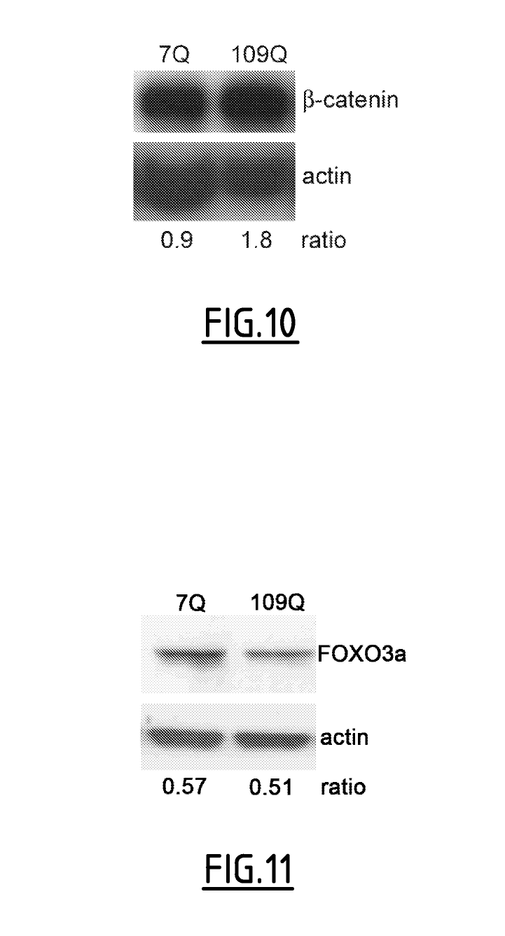

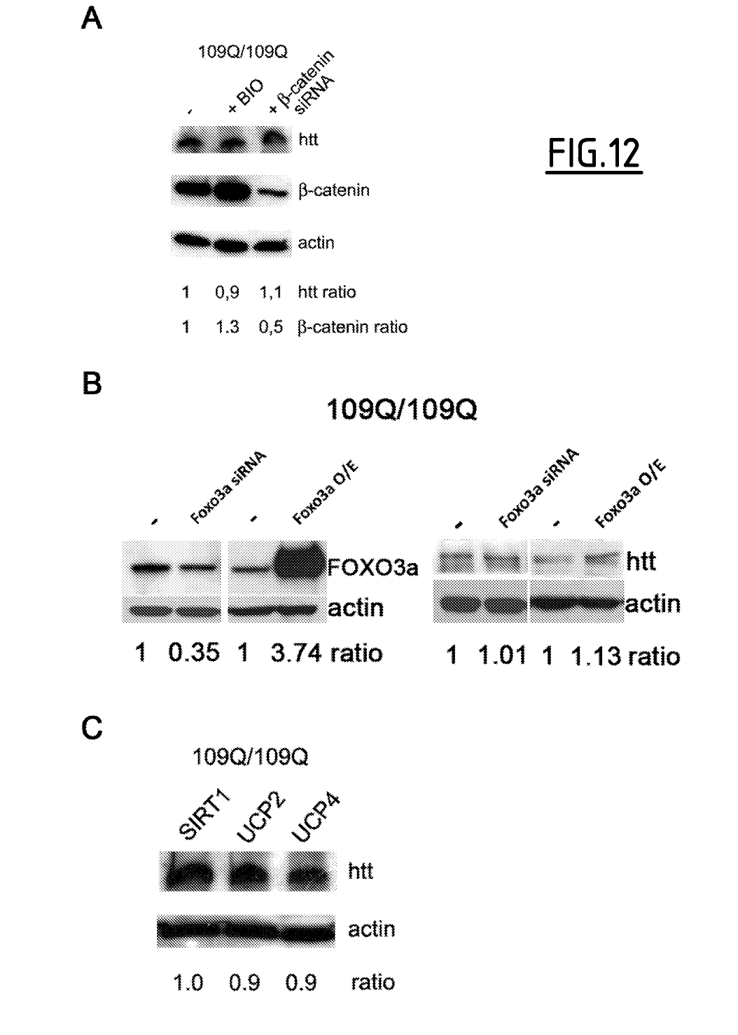

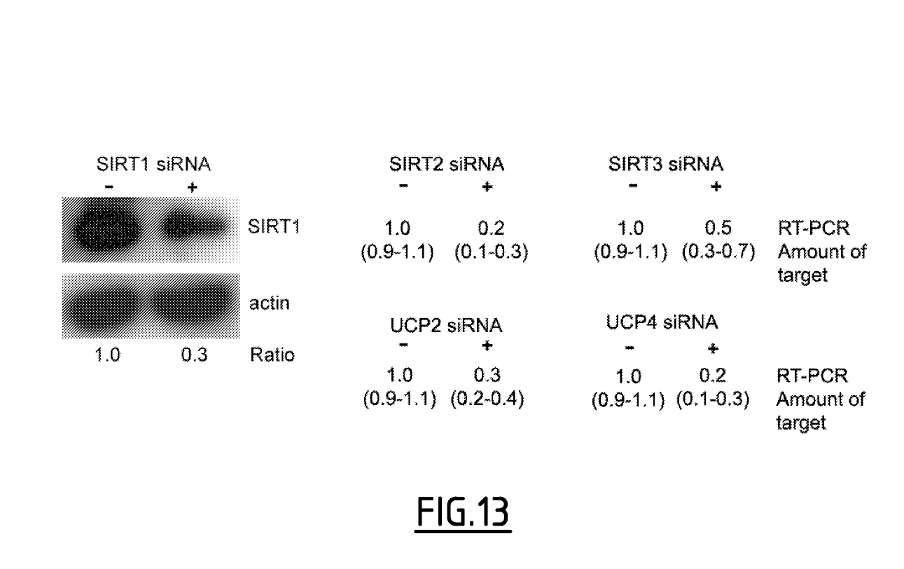

Having shown that a gene network centered onto daf-16 modulates the neurotoxicity of a N-terminal htt fragment in nematodes, this network was next investigated in striatal cells from the full length htt knock-in mice HdhQ111 (Trettel et al. 2000 Hum Mol Genet. 9:2799-2809). Mutant htt (109Q/109Q) striatal cells have a higher rate of cell death induced by serum deprivation compared to wild-type (7Q/7Q) cells (Trettel et al. 2000 Hum Mol Genet. 9:2799-2802), thus providing an assay to test for mammalian cell vulnerability to mutant htt expressed at endogenous levels. As previously reported, mutant htt cells showed increased β-catenin levels (FIG. 10) which may correspond to a pro-survival response. Additionally, β-catenin siRNA decreased the survival of 109Q/109Q cells (FIG. 3A/3B). There was no change in htt expression, and β-catenin was reduced (FIG. 12A). The GSK-3β inhibitor BIO enhanced the survival of 109Q/109Q cells (FIG. 3B), an effect accompanied by increased β-catenin (FIG. 12A) and lost by β-catenin siRNA (FIG. 3B). Next, the role of FoxO3a, a mammalian homolog of DAF-16 involved in neuroprotection was tested. In contrast to β-catenin, FoxO3a levels were identical in normal and mutant htt cells (FIG. 12B). FoxO3a siRNA decreased the survival of 109Q/109Q cells (FIG. 3C) with a concomitant decrease of FoxO3a expression (FIG. 12C). Conversely, FoxO3a overexpression increased 109Q/109Q cell survival (FIG. 3D) with increased FoxO3a expression (FIG. 12C). The expression of htt was unchanged by reducing or increasing FoxO3a (FIG. 12C). SIRT1 siRNA reduced SIRT1 (FIG. 13) but had no effect on cell survival (FIG. 3D). While siRNA against SIRT2 or SIRT3 reduced target mRNA levels (FIG. 13), they had no effect on cell survival. UCP2 siRNA decreased the survival of 109Q/109Q cells (FIG. 3D), with no change in htt expression (FIG. 12C) and decreased UCP2 mRNA levels (FIG. 13). UCP4 siRNA increased the survival of 109Q/109Q cells (FIG. 3D), with no change in htt expression (FIG. 12) and decreased UCP4 mRNA levels (FIG. 13). These results suggested evolutionary diversity for mouse UCP activity compared to the sole UCP in C. elegans.

### Example 5

### Synergy of β-catenin and SIRT1 in Mutant htt Striatal Cells

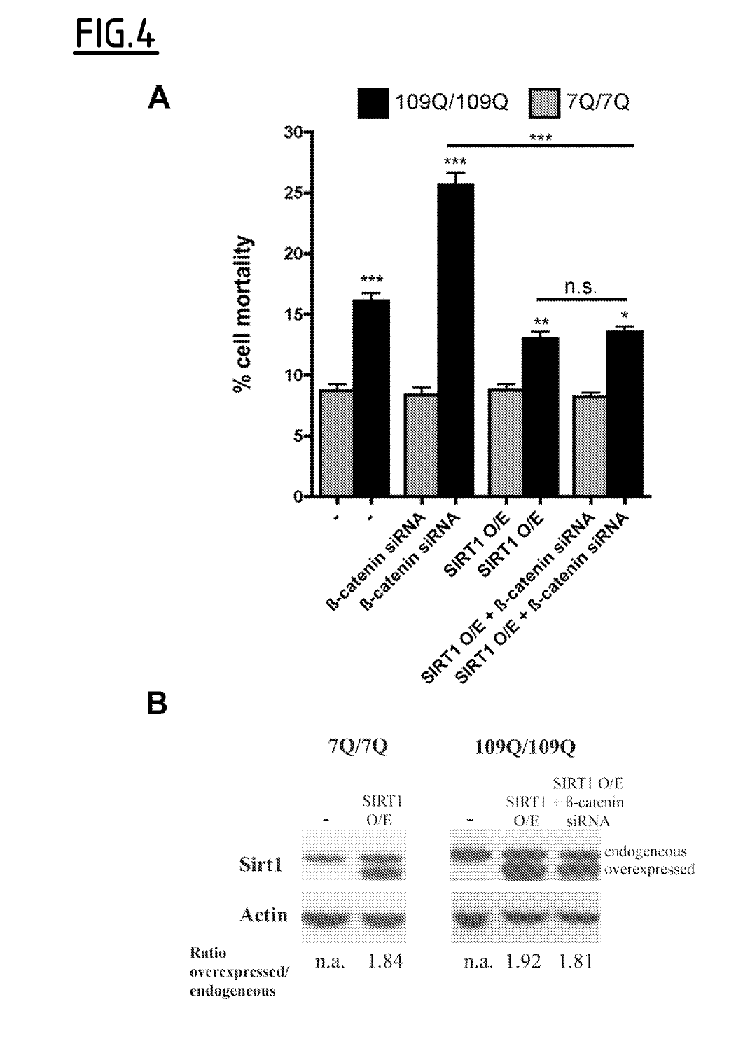

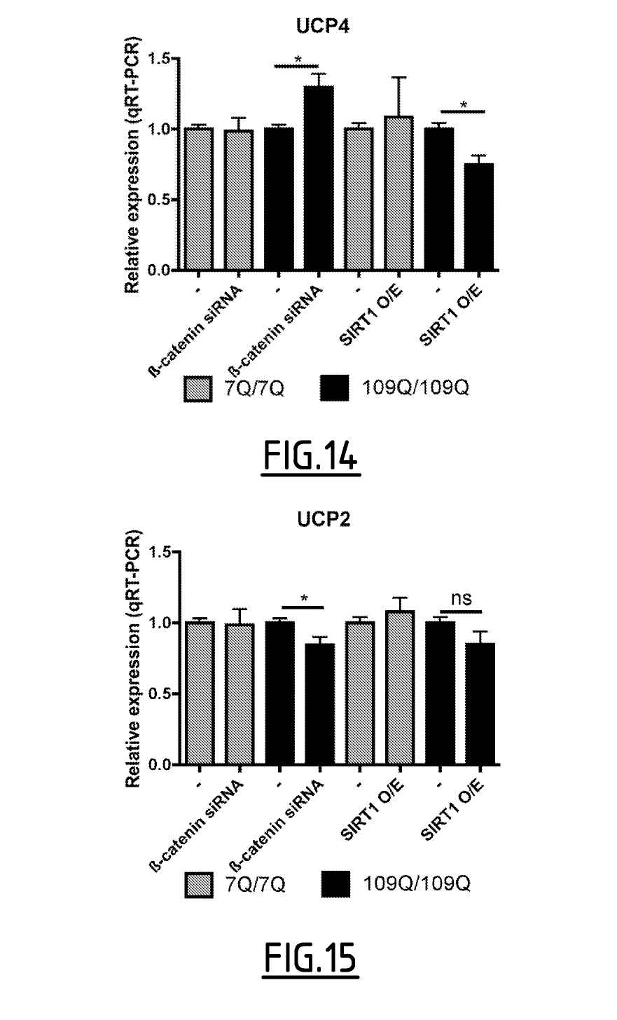

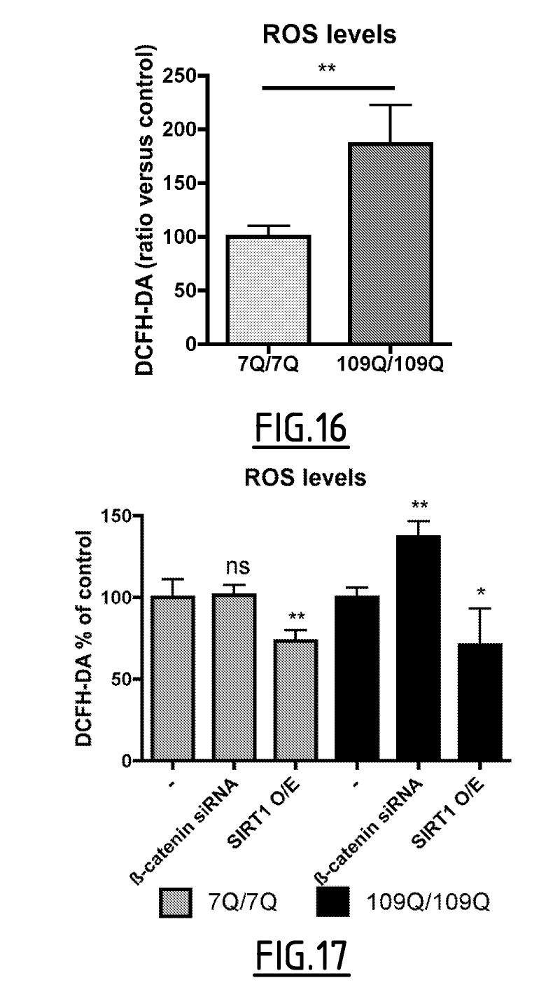

β-catenin and SIRT1 regulate FoxO activity and the UCP-4 promoter is regulated by DAF-16/FoxO in nematodes, suggesting that β-catenin and SIRT1 may act together on neuron survival, which may involve an effect the expression of on neuronal UCPs such as UCP2 and UCP4. SIRT1 overexpression was neuroprotective and suppressed the reduction of mutant htt striatal cell survival elicited by β-catenin siRNA (FIG. 4), highlighting the importance of a synergy of β-catenin and SIRT1 on mutant htt cell survival. Additionally, it was observed that, in mutant htt cells, reducing the levels of β-catenin, a neuroprotective factor, increased mRNA levels of UCP4 (detrimental to cell survival) while the overexpression of SIRT1, which may be protective as suggested by sir-2.1/SIRT1 activity in polyQ nematodes, had the opposite effect (FIG. 14). Conversely, β-catenin siRNA decreased UCP2 mRNA levels of UCP2 (a neuroprotective gene) in mutant htt cells with however no significant effect of SIRT1 overexpression (FIG. 15). These results suggested that β-catenin is a more specific regulator of neuron survival compared to SIRT1. β-catenin siRNA increased reactive oxygen species (ROS) levels in mutant htt striatal cells whereas SIRT1 overexpression decreased ROS levels in mutant and normal htt cells (FIGS. 16 and 17), further suggesting that β-catenin is a tight regulator of neuron survival.

### Example 6

### Effects of the GSK-313, TCERG1, FZD10 and FoxO1 Genes on Age-at-Onset of HD in European Patients

To explore whether polymorphism in genes of the FoxO network may modify AAO of HD, the effects of genetic variants was tested in the GSK-3β, TCERG1, FZD10, FoxO1, FoxO3a and UCP2 genes on age-at-onset (AAO) of first symptoms (motor, cognitive or neuropsychiatric symptoms) of HD in the French cohort (347 patients). Single nucleotide polymorphism (SNP) markers were selected based on their previously-described associations to human pathology (see Materials and methods). Additionally, the Gln-Ala repeat variant in the transcriptional regulator TCERG1 was evaluated. It was elected to include TCERG1 in this analysis because it protects from mutant htt, TCERG1 is coded by a conserved gene that localizes to a susceptibility locus for HD, is a putative HD modifier and is known as a daf-16 interactor and lifespan modulator in nematodes. Patients with or without FoxO3a or UCP2 variants had similar AAO. In contrast, the polymorphic Gln-Ala repeat in TCERG1 modified AAO of HD (Table 5). This corresponded to a trend towards a later mean AAO (for example, +4 years for all patients) contributed by genotypes with short repeat alleles (281 bp, 287 bp) compared to genotypes with other (298 bp, 310 bp) repeat alleles, consistent with previous studies indicating that American patients carrying long repeat alleles have an earlier AAO of HD (Holbert et al. 2001 Proc Natl Acad Sci USA 98:1811-1816). Additionally, a SNP in the promoter of GSK-3β (rs334558) modified AAO only in those patients with motor onset (Table 5). This corresponded to a trend towards a later mean AAO (+3 years) contributed by less frequent genotypes. Finally, GSK-3β appeared to modify the AAO of HD (all first symptoms) together with TCERG1, notably in 326 patients with frequent mutant htt alleles (Table 5).

TABLE 5
Regression analysis of htt, TCERG1 and GSK-3β against AAO of HD in
the French cohort

| | | | | |
|---|---|---|---|---|
| Regression analysis | Variables | R² | ΔR² | P value |

All patients (n = 347)

| | | | | |
|---|---|---|---|---|
| 1 | Htt | 0.615 | — | <0.001 |
| 2 | Htt + TCERG1 | 0.623 | 0.008 | 0.004 |
| 3 | Htt + GSK-3β | 0.615 | 0.000 | 0.586 |
| 4 | Htt + [TCERG1-GSK-3β] | 0.623 | 0.008 | 0.016 |

Patients with 38 < (CAG)n < 53 (n = 326)

| | | | | |
|---|---|---|---|---|
| 5 | Htt | 0.520 | — | <0.001 |
| 6 | htt + TCERG1 | 0.529 | 0.009 | 0.009 |
| 7 | htt + GSK-3β | 0.520 | 0.000 | 0.461 |
| 8 | htt + [TCERG1-GSK-3β] | 0.530 | 0.010 | 0.021 |

Patients with motor symptoms at onset: effect of htt and
TCERG1 (n = 137)

| | | | | |
|---|---|---|---|---|
| 9 | htt | 0.739 | — | <0.001 |
| 10 | htt + TCERG1 | 0.749 | 0.010 | 0.012 |

Patients with motor symptoms at onset: effect of htt and GSK-3β
(n = 153)

| | | | | |
|---|---|---|---|---|
| 11 | Htt | 0.742 | — | <0.001 |
| 12 | htt + GSK-3β | 0.750 | 0.008 | 0.016 |

Patients with motor symptoms at onset: effect of htt, TCERG1 and
GSK-3β (n = 130)

| | | | | |
|---|---|---|---|---|
| 13 | htt | 0.756 | — | <0.001 |
| 14 | htt + [TCERG1-GSK-3β] | 0.768 | 0.012 | 0.024 |

A linear regression model was used that uses larger htt allele, TCERG1 and GSK-313 genotypes as independent variables, and log AAO as a dependent variable. Two groups were used for TCERG1 genotypes, one group for small rare repeat alleles (281 or 287 bps) and one group for frequent (298 bp) and large rare (310 bp) repeat alleles. Two groups were used for GSK-3β promoter genotypes (SNP rs334558), one group for the most frequent AA and AG genotypes (AA and AG), and one group for rare genotypes (GG). When TCERG1 is added as a second independent variable, an increase in R² (ΔR²) was detected for the entire cohort (analysis 2). The effect is still significant for patients that carry frequent mutant htt alleles (analysis 6) and those with motor symptoms at onset (analysis 10). When GSK-3β is added as a second independent variable, an increase in R² was detected for HD patients with motor symptoms (analysis 12). When both TCERG1 and GSK-3β are added as one grouped independent variable (see SI Materials and methods), an equivalent increase in R² was detected compared to TCERG1 alone in the entire cohort (analysis 4). This effect was more pronounced in the more homogeneous cohort that carries frequent mutant htt alleles (analysis 8). An increase was also detected in patients with motor symptoms at onset (analysis 14), which is however subjected to caution due to the small number of samples available, does not apply.

Next, it was expanded on the number of markers examined in the GSK-3β and TCERG1 genes. To this end, 13 SNPs from GSK-3β and 11 SNPs from TCERG1 were selected to provide full coverage of the polymorphisms that may arise in these genes. It was also tested whether the associations in the French cohort held true in a wider European population. The selected SNPs was thus examined for their effects in 538 REGISTRY participants (European HD network observational study) from several countries other than France. SNPs of all genes were selected in modeling AAO in an automatic model selection procedure. SNPs from GSK-3β or TCERG1 modified AAO of HD (Table 7), notably in patients with motor symptoms at onset (Table 6). SNPs from FoxO1 and/or FZD10 (Table 8) and GSK-3β and/or FZD10 (Table 9) were also shown to modify age at onset. The presence of rare alleles in GSK-3β SNP rs11925899 corresponded to an earlier AAO (see legend of Table 7); in contrast, rare alleles in TCERG1 SNPs rs3756502, rs7731904, rs1978708, rs2400220, rs2241697 and rs12519022 were associated with a delayed AAO. Based on the model containing SNPs from both GSK-3β and TCERG1 in patients with motor onset only, 95% prediction intervals were computed for two hypothetical individuals with a mutant htt allele of 45 CAG repeats. In the worst case (no rare alleles for rs9851174 and rs12519022, two rare alleles for rs17811013 and rs11925899), the predicted AAO was 32.4 (95% prediction interval [22.5, 46.6]). In the best case (two rare alleles for rs9851174 and rs12519022, no rare alleles for rs17811013 and rs11925899), the predicted AAO was 47.4 (95% prediction interval [33.3, 67.4]). Together, these results highlighted GSK-3β as a modifier of AAO of first symptoms in HD and pointed towards additive effects with TCERG1 and also highlighted FoxO1 and FZD10 as modifiers of AAO of first symptoms in HD.

TABLE 6
Regression analysis of htt, GSK-3β and TCERG1 against AAO of HD in
the European cohort

| Model Input | Model | R² | ΔR² | P value |
|---|---|---|---|---|
| htt | 6.3066 − 0.0577 · htt | 0.5965 | — | — |
| htt + GSK-3β | 6.3276 − 0.0578 · htt − 0.0574 · rs11925899_{GSK-3b} | 0.6060 | 0.0095 | 0.0065 |
| htt + TCERG1 | 6.2723 − 0.0574 · htt + 0.0319 · rs2241697_{TCERG1} | 0.6012 | 0.0047 | 0.0422 |
| htt + GSK-3β + | 6.2555 − 0.0570 · htt − 0.0445 · rs11925899_{GSK-3b} + | 0.6160 | 0.0195 | 0.0018 |
| TCERG1 | 0.0384 · rs12519022_{TCERG1} + | | | |
| | 0.0448 · rs9851174_{GSK-3b} − | | | |
| | 0.0628 · rs17811013_{GSK-3b} | | | |

Linear regression with forward selection was used for modeling. This table shows the data only for 272 patients with motor symptoms at onset (See Table 7 for the full analysis and for comments). The model using htt alone is detailed, then the final models based on SNPs in GSK-3B, TCERG1 and both are listed as well as their respective adjusted R² in the model, the difference in R² to the model containing htt only and the P value of the likelihood ratio test comparing the respective model to the model containing htt only.

TABLE 7
Full data for regression analysis of htt, GSK-3β and TCERG1 against
AAO of HD in the European cohort

| | | | | | |
|---|---|---|---|---|---|
| Regr. | Model | | | | |
| analysis | Input | Model | R² | ΔR² | P value |

All patients (n = 528)

| | | | | | |
|---|---|---|---|---|---|
| 1 | htt | 6.0722 − 0.0532 · htt | 0.4815 | — | — |
| 2 | htt + GSK- | 6.0798 − 0.0531 · htt − 0.0396 · rs11925899_{GsK-3b} | 0.4854 | 0.0039 | 0.0262 |
| | 3β | | | | |
| 3 | htt + | 6.0461 − 0.0530 · htt + 0.0286 · rs3756502_{TCERG1} + | 0.4847 | 0.0032 | 0.0736 |
| | TCERG1 | 0.0387 · rs7731904_{TCERG1} | | | |
| 4 | htt + GSK- | 6.0530 − 0.0529 · htt − 0.0418 · rs11925899_{GSK-3b} + | 0.4889 | 0.0074 | 0.0147 |
| | 3β + | 0.0296 · rs3756502_{TCERG1} + | | | |
| | TCERG1 | 0.0405 · rs1978708_{TCERG1} | | | |

Patients with 38 < (CAG)n < 53 (n = 508)

| | | | | | |
|---|---|---|---|---|---|
| 5 | htt | 6.4630 − 0.0620 · htt | 0.4234 | — | — |
| 6 | htt + GSK- | 6.4659 − 0.0619 · htt − 0.0351 · rs11925899_{GSK-3b} | 0.4267 | 0.0033 | 0.0471 |
| | 3β | | | | |
| 7 | htt + | 6.4283 − 0.0616 · htt + 0.0226 · rs3756502_{TCERG1} + | 0.4262 | 0.0028 | 0.1074 |
| | TCERG1 | 0.0396 · rs7731904_{TCERG1} | | | |
| 8 | htt + GSK- | 6.4471 − 0.0618 · htt − 0.0362 · rs11925899_{GSK-3b} + | 0.4284 | 0.0050 | 0.0410 |
| | 3β + | 0.0201 · rs2400220_{TCERG1} | | | |
| | TCERG1 | | | | |

Patients with motor symptoms at onset (n = 272)

| | | | | | |
|---|---|---|---|---|---|
| 9 | htt | 6.3066 − 0.0577 · htt | 0.5965 | — | — |
| 10 | htt + GSK- | 6.3276 − 0.0578 · htt − 0.0574 · rs11925899_{GSK-3b} | 0.6060 | 0.0095 | 0.0065 |
| | 3β | | | | |
| 11 | htt + | 6.2723 − 0.0574 · htt + 0.0319 · rs2241697_{TCERG1} | 0.6012 | 0.0047 | 0.0422 |
| | TCERG1 | | | | |
| 12 | htt + GSK- | 6.2555 − 0.0570 · htt − 0.0445 · rs11925899_{GSK-3b} + | 0.6160 | 0.0195 | 0.0018 |
| | 3β + | 0.0384 · rs12519022_{TCERG1} + | | | |
| | TCERG1 | 0.0448 · rs9851174_{GSK-3b} − | | | |
| | | 0.0628 · rs17811013_{GSK-3b} | | | |

Linear regression with forward selection was used for modeling. This table summarizes the results. For each population inspected, first the model using htt alone is detailed, then the final models based on the SNPs in GSK-3B, TCERG1 and both are listed as well as their respective adjusted R² in the model, the difference in R² to the model containing htt only and the P value of the likelihood ratio test comparing the respective model to the model containing htt only. The similarity between analyses 1 to 4 and 5 to 8 is to a large extend explained by the nearly identical populations they are based upon. The SNP rs11925899 of GSK-3β entered all models for which it was available as independent variable (analyses 2,4,6,8,10 and 12), indicating that it is quite a strong factor. Gains in R² are between 0.003 and 0.010 for the models containing only GSK-3β and the parameter estimates for rs11925899 vary between −0.0351 (analysis 6) and −0.0574 (analysis 10). This indicates that AAO decreases with each additional rare alleles ('A′ in this case). E.g., for analysis 10, it follows that each additional rare allele leads to a factor of e⁻⁰⁰⁵⁷⁴=0.9442 for the AAO to be multiplied with. If, e.g., a patient with no rare allele in rs11925899 had predicted AAO of 50, one rare allele would lead to a decrease in prediction to 47.2 and two rare alleles would lead to a prediction of 44.6. Likelihood ratio test p-values for all models containing SNPs from GSK-3β are significant at an a-level of 0.05. Analyses 3 and 7 both lead to inclusion of the two SNPs rs3756502 and rs7731904 in the TCERG1 gene, which both have a positive effect on AAO, but both models are not significant at an a-level of 0.05 in the likelihood ratio test. In other models, different SNPs of TCERG1 were included by the model selection procedure.

TABLE 8
Regression analysis of htt, FOXO1 and FZD10 against AAO of HD in the
European cohort.

| | | | | | |
|---|---|---|---|---|---|
| Regr. | | | | | |
| analysis | Model Input | Model | R² | ΔR² | p-value* |

All patients (n = 528)

| | | | | | |
|---|---|---|---|---|---|
| 1 | htt | 6.0601 − 0.0529 · htt | 0.4809 | — | — |
| 2 | htt + FOXO1 | 6.0641 − 0.0530 · htt + 0.0299 · rs17446593 − | 0.4845 | 0.0036 | 0.0597 |
| | | 0.0232 · rs12865518 | | | |
| 3 | htt + FZD10 | 6.0184 − 0.0535 · htt + 0.0497 · rs7953082 + | 0.4956 | 0.0147 | 0.0012 |
| | | 0.1020 · rs10848026 + 0.0331 · rs3741568 + | | | |
| | | 0.0484 · rs7966482 − | | | |
| | | 0.0850 · rs10848026 × rs7966482 | | | |
| 4 | htt + FOXO1 + | 6.0595 − 0.0538 · htt + 0.0452 · rs17446593 + | 0.5085 | 0.0276 | 3.2 · 10⁻⁵ |
| | FZD10 | 0.0509 · rs7953082 + 0.1184 · rs10848026 − | | | |
| | | 0.0076 · rs3741568 + 0.0167 · rs7966482 − | | | |
| | | 0.0790 · rs7981045 − | | | |
| | | 0.0468 · rs17446593 × rs10848026 − | | | |
| | | 0.0830 · rs10848026 × rs7966482 + | | | |
| | | 0.0757 · rs3741568 × rs7981045 + | | | |
| | | 0.0591 · rs7966482 × rs7981045 | | | |

Patients with 38 < (CAG)n < 53 (n = 508)

| | | | | | |
|---|---|---|---|---|---|
| 5 | htt | 6.4356 − 0.0614 · htt | 0.4217 | — | — |
| 6 | htt + FOXO1 | 6.4272 − 0.0612 · htt + 0.0262 · rs17446593 − | 0.4249 | 0.0032 | 0.0918 |
| | | 0.0220 rs12865518 | | | |
| 7 | htt + FZD10 | 6.3953 − 0.0620 · htt + 0.0468 · rs7953082 + | 0.4373 | 0.0156 | 0.0022 |
| | | 0.0995 · rs10848026 + 0.0354 · rs3741568 + | | | |
| | | 0.0325 · rs7966482 − | | | |
| | | 0.0786 · rs10848026 × rs7966482 | | | |
| 8 | htt + FOXO1 + | 6.3997 − 0.0619 · htt + 0.0290 · rs17446593 + | 0.4434 | 0.0217 | 0.0007 |
| | FZD10 | 0.0467 · rs7953082 − 0.0364 · rs12865518 + | | | |
| | | 0.0994 · rs10848026 + 0.0082 · rs7966482 + | | | |
| | | 0.0359 · rs3741568 − | | | |
| | | 0.0813 · rs10848026 × rs7966482 + | | | |
| | | 0.0549 · rs12865518 × rs7966482 | | | |

Patients with motor symptoms at onset (n = 272)

| | | | | | |
|---|---|---|---|---|---|
| 9 | htt | 6.2932 − 0.0574 · htt | 0.5969 | — | — |
| 10 | htt + FOXO1 | 6.2932 − 0.0574 · htt | 0.5969 | 0.0000 | — |
| 11 | htt + FZD10 | 6.3815 − 0.0586 · htt − 0.0895 · rs2078105 − | 0.6133 | 0.0164 | 0.0014 |
| | | 0.0376 rs3741568 | | | |
| 12 | htt + FOXO1 + | 6.3815 − 0.0586 · htt − 0.0895 · rs2078105 − | 0.6133 | 0.0164 | 0.0014 |
| | FZD10 | 0.0376 · rs3741568 | | | |

Together, these results highlighted FOXO1 and FZD10 as modifiers of AAO of motor symptoms in HD and pointed towards additive effects with TCERG1.

TABLE 9
Regression analysis of htt, FZD10 and GSK-3β against AAO of HD in the
European cohort.

| | | | | | |
|---|---|---|---|---|---|
| Regr. | | | | | p- |
| analysis | Model Input | Model | R² | ΔR² | value* |

All patients (n = 526)

| | | | | | |
|---|---|---|---|---|---|
| 1 | htt | 6.0749 − 0.0532 · htt | 0.4827 | — | — |
| 2 | htt + FZD10 | 5.9962 − 0.0537 · htt + 0.1505 · rs10848026 + | 0.4986 | 0.0159 | 0.0016 |
| | | 0.0261 · rs11060759 + 0.0155 · rs7966482 + | | | |
| | | 0.0608 · rs7953082 + 0.0469 · rs3741568 − | | | |
| | | 0.0420 · rs10848026:rs11060759 − | | | |
| | | 0.0575 rs10848026:rs7966482 | | | |
| 3 | htt + GSK3-β | 6.0826 − 0.0531 · htt − 0.0399 · rs11925899 | 0.4867 | 0.0040 | 0.0255 |
| 4 | htt + FZD10 + | 5.9737 − 0.0534 · htt + 0.0118 · rs11925899 + | 0.5041 | 0.0214 | 0.0003 |
| | GSK3-β | 0.1442 · rs10848026 + 0.0515 · rs11060759 + | | | |
| | | 0.0189 · rs7966482 + 0.0612 · rs7953082 + | | | |
| | | 0.0475 · rs3741568 − | | | |
| | | 0.0457 · rs10848026 × rs11060759 − | | | |
| | | 0.0550 · rs11925899 × rs11060759 − | | | |
| | | 0.0558 · rs10848026 × rs7966482 | | | |

Patients with 38 < (CAG)n < 53 (n = 506)

| | | | | | |
|---|---|---|---|---|---|
| 5 | htt | 6.4665 − 0.0621 · htt | 0.4248 | — | — |
| 6 | htt + FZD10 | 6.4760 − 0.0622 · htt − 0.0351 · rs2078105 | 0.4269 | 0.0021 | 0.0938 |
| 7 | htt + GSK3-β | 6.4695 − 0.0619 · htt − 0.0353 · rs11925899 | 0.4282 | 0.0034 | 0.0458 |
| 8 | htt + FZD10 + | 6.4774 − 0.0620 · htt − 0.0319 · rs11925899 − | 0.4294 | 0.0046 | 0.0497 |
| | GSK3-β | 0.0300 rs2078105 | | | |

Patients with motor symptoms at onset (n = 272)

| | | | | | |
|---|---|---|---|---|---|
| 9 | htt | 6.3065 − 0.0577 · htt | 0.5965 | — | — |
| 10 | htt + FZD10 | 6.4294 − 0.0596 · htt − 0.1170 · rs2078105 − | 0.6395 | 0.0430 | 4.8 · 10⁻⁶ |
| | | 0.0428 · rs3741568 − 0.0063 · rs11060759 + | | | |
| | | 0.1493 · rs10848026 − 0.0568 · rs7966482 + | | | |
| | | 0.1783 · rs2078105 × rs10848026 − | | | |
| | | 0.0886 · rs11060759 × rs10848026 | | | |
| 11 | htt + GSK3-β | 6.3275 − 0.0578 · htt − 0.0574 · rs11925899 | 0.6060 | 0.0095 | 0.0065 |
| 12 | htt + FZD10 + | 6.4008 − 0.0587 · htt − 0.0807 · rs2078105 − | 0.6184 | 0.0219 | 0.0005 |
| | GSK3-β | 0.0432 · rs11925899 − 0.0347 · rs3741568 | | | |

### C. Conclusion

These data indicate that β-catenin interacts with sir-2.1 in nematode neurons and SIRT1 in mouse neurons to protect from expanded polyQ/htt. This effect involves UCPs since DAF-16 regulates the promoter of the sole UCP in C. elegans and expression of neuronal UCP such as UCP2/UCP4 is regulated by β-catenin, SIRT1, or both. UCPs can have profound effects on neurons, with chronic uncoupling promoting mitochondrial biogenesis and elevated ATP. The enhancement of 128Q toxicity in nematode ucp-4 mutants suggests that chronic uncoupling is beneficial to HD neurons. In mouse models of Parkinson's disease, increased uncoupling from UCP2 overexpression is neuroprotective. Consistently, UCP2 protected 1090/1090 striatal cells from cell death, and β-catenin promoted UCP2 expression. UCP4 appears to act oppositely to UCP2, which may be due to turn over differences.

These data also show that polymorphisms in the genes involved in the FoxO network, in particular in GSK-3β gene or the TCERG1 gene, modify the age-at-onset of motor symptoms in HD patients.

The activation of cell survival mechanisms controlled by FoxO may contribute to slowing down HD pathogenesis. These data indicate that GSK-3β inhibitors require the FoxO network for neuroprotection, suggesting that FoxO-dependent protection in the HD brain may be elicited by lithium. This drug has GSK-3β inhibition activity and is active in a mouse model of HD , which together with the data presented herein suggest that GSK-3β has significant potential for the treatment and prognosis of HD.

In conclusion, these data reveal that GSK3/β-catenin acts together with SIRT1 on HD neuron survival. Additionally, these data identify several genes as modifiers of age-at-onset of motor symptoms in HD.

1-11. (canceled)

12. A method of genotyping comprising the steps of:
a) obtaining an isolated nucleic acid from a biological sample derived from a single individual; and
b) detecting the nucleotide present at:
i one or more GSK-3β biallelic markers; and/or
ii one or more TCERG1-related biallelic markers; and/or
iii one or more FoxO1 biallelic markers; and/or
iiii one or more FZD10 biallelic markers; and
c) optionally determining the length of htt gene GAG repeats.

13. The method according to claim 12, wherein said GSK-3β related biallelic markers are selected from the group of biallelic markers shown in the table set forth in claim 22, and wherein said TCERG1-related biallelic markers are selected from the group of biallelic markers shown in the table set forth in claim 23.

14. The method according to claim 13, wherein the presence of:
i. no rare allele at biallelic marker NO: 1 (rs9851174);
ii. no rare allele at biallelic marker NO: 19 (rs12519022);
iii. two rare alleles at biallelic marker NO: 2 (rs17811013); and
iv. two rare alleles at biallelic marker NO: 3 (rs11925899);
indicates that said individual is likely to develop symptoms of Huntington's disease at an early age.

15. The method according to claim 13, wherein the presence of:
i. two rare alleles at biallelic marker NO: 1 (rs9851174);
ii. two rare alleles at biallelic marker NO: 19 (rs12519022);
iii. no rare allele at biallelic marker NO: 2 (rs17811013); and
iv. no rare allele at biallelic marker NO: 3 (rs11925899);
indicates that said individual is likely to develop symptoms of Huntington's disease at a later age.

16. A kit comprising:
a) means for detecting the nucleotide present at:
i one or more GSK-3β related biallelic markers; and/or
ii one or more TCERG1 related biallelic markers; and/or
iii one or more FoxO1 biallelic markers; and/or
iv one or more FZD10 biallelic markers;
b) instructions for use in the prognosing the age-at-onset of symptoms of Huntington's disease; and, optionally,
c) one or more reagents.

17-19. (canceled)

20. A method for prognosing the age-at-onset of symptoms of Huntington's disease in an individual at risk of suffering from said disease, which method comprises detecting at least one gene related SNP biallelic marker, wherein said gene related SNP biallelic marker is a biallelic marker from a gene selected from the group consisting of: GSK-3β, TCERG1, FoxO1, FZD10, adducin ADD2, β-catenin, SOD1, SOD2, SOD3, ANK2, PRKAA1, TCERG1, FYN, SGK1, DKK1, SIRT1, SIRT2, SIRT3, UCP1, UCP2, UCP4, GABARAPL1, PRKAB1, RYK, FoxO3A, FOXA1, AKT1, PIK3R2, and LIG1.

21. The method according to claim 20, wherein said gene is GSK-3β and/or TCERG1 and/or FoxO1 and/or FDZ10.

22. The method according to claim 20, wherein said GSK-3β related biallelic marker is selected from the group consisting of the GSK-3β related biallelic markers shown in the table below:

| Biallelic marker No. | Position | in SEQ ID No. | Alternatives alleles |
|---|---|---|---|
| 1 | 27 | 1 | A/G |
| 2 | 27 | 2 | C/G |
| 3 | 27 | 3 | A/C |
| 4 | 27 | 4 | G/T |
| 5 | 27 | 5 | A/G |
| 6 | 27 | 6 | A/G |
| 7 | 27 | 7 | C/T |
| 8 | 27 | 8 | A/G |
| 9 | 27 | 9 | A/G |
| 10 | 27 | 10 | A/G |
| 11 | 27 | 11 | C/G |
| 12 | 27 | 12 | A/G |
| 13 | 27 | 13 | A/T |

23. The method according to claim 20, wherein said at least one TCERG1 related biallelic marker is selected from the group consisting of the TCERG1 related biallelic markers shown in the table below:

| Biallelic marker | | in SEQ ID | |
|---|---|---|---|
| No. | Position | No. | Alternatives alleles |
| 14 | 27 | 14 | A/G |
| 15 | 27 | 15 | C/T |
| 16 | 27 | 16 | A/G |
| 17 | 27 | 17 | C/T |
| 18 | 27 | 18 | A/G |
| 19 | 27 | 19 | A/G |
| 20 | 27 | 20 | C/T |
| 21 | 27 | 21 | C/T |
| 22 | 27 | 22 | G/T |
| 23 | 27 | 23 | C/G |
| 24 | 27 | 24 | C/T |

24. The method according to claim 20, wherein said at least one FoxO1 related biallelic marker is selected from the group consisting of the FoxO1 related biallelic markers shown in the table below:

| Biallelic marker | | in SEQ ID | Alternatives |
|---|---|---|---|
| No. | Position | No. | alleles |
| 25 | 27 | 70 | A/G |
| 26 | 27 | 71 | A/G |
| 27 | 27 | 72 | A/G |
| 28 | 27 | 73 | A/G |

25. The method according to claim 20, wherein said at least one FZD10 related biallelic marker is selected from the group consisting of the FZD10 related biallelic markers shown in the table below:

| Biallelic marker | | in SEQ ID | Alternatives |
|---|---|---|---|
| No. | Position | No. | alleles |
| 29 | 27 | 74 | A/T |
| 30 | 27 | 75 | C/G |
| 31 | 27 | 76 | A/G |
| 32 | 27 | 77 | C/G |
| 33 | 27 | 78 | G/T |
| 34 | 27 | 79 | A/T |

26. The method according to claim 20, wherein a combination of at least one GSK-33 related biallelic marker and at least one TCERG1 related biallelic marker is detected.

27. The method according to claim 26, wherein said combination is selected from the group consisting of biallelic markers Nos. 3, 14 and 16, biallelic markers Nos. 3 and 17, and biallelic markers Nos. 1, 2, 3 and 19.

28. The method according to claim 20, wherein a combination of at least one FoxO1 related biallelic marker and at least one FZD10 related biallelic marker is detected.

29. The method according to claim 20, wherein a combination of at least one GSK-33 related biallelic marker and at least one FZD10 related biallelic marker is detected.

30. The method according to claim 20, wherein the method further comprises detecting the length of htt gene GAG repeats.

31. The method according to claim 12, wherein the method further comprises a step d) of correlating the result of the genotyping steps with the age at onset of symptoms of Huntington's disease.

32. A method for treating or preventing Huntington's disease, wherein said method comprises administering to a subject in need thereof lithium or a salt thereof at a dose inferior to 10 mg per day.

33. The method according to claim 32, wherein said lithium or salt thereof is administered at a dose within the range of 100 μg to 1 mg per day.

34. The method according to claim 32, wherein said lithium or salt thereof is lithium carbonate.
